(19) United States
(12) Patent Application Publication
(10) Pub. No.: US 2025/0228848 A1
(43) Pub. Date: Jul. 17, 2025

(54) PROCESS FOR MAKING A KRAS G12C INHIBITOR

(71) Applicant: ELI LILLY AND COMPANY, Indianapolis, IN (US)

(72) Inventors: Serge Louis BOULET, Fishers, IN (US); Lee Joseph BURNS, Indianapolis, IN (US); Kevin Paul COLE, Indianapolis, IN (US); Xueqian GONG, Carmel, IN (US); Deqi GUO, Carmel, IN (US); David Michael HYMAN, Stamford, CT (US); Michael Edward LAURILA, Mooresville, IN (US); Ryan James LINDER, Carmel, IN (US); Sheng-Bin PENG, Carmel, IN (US); James Craig RUBLE, Greenwood, IN (US); Chong SI, Zionsville, IN (US); Hannah Leslie VONESH, Carmel, IN (US)

(21) Appl. No.: 18/853,699

(22) PCT Filed: Apr. 7, 2023
(86) PCT No.: PCT/US2023/065509

§ 371 Date: Oct. 2, 2024

Related U.S. Application Data
(60) Provisional application No. 63/492,119, filed on Mar. 24, 2023.
(60) Provisional application No. 63/328,520, filed on Apr. 7, 2022.

(51) Int. Cl. A61K 31/4985; A61P 35/00; C07D 241/04; C07D 498/04

(52) U.S. Cl. CPC A61K 31/4985; A61P 35/00; C07D 241/04; C07D 498/04

(57) ABSTRACT

The present disclosure provides the compound of Formula I:

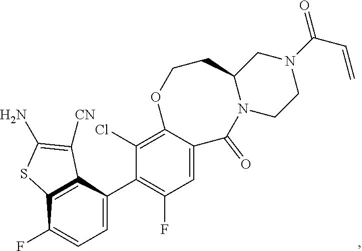

or pharmaceutically acceptable salts thereof, methods and intermediates useful in preparing the compound of Formula I, pharmaceutical compositions containing the compound of Formula I, and methods of treating cancer using the compound of Formula I.

The present disclosure relates to compounds, and pharmaceutically acceptable salts thereof that can be used to treat cancer.

WO 2021/118877 and US 2021/0179633 A1 (hereinafter “the '877 and '633 references”) disclose certain KRas G12C inhibitors, or salts thereof, that can be used to treat cancer. Methods of preparing these compounds are also disclosed. The compounds made using the methods disclosed in the '877 and '633 references may contain impurities. These impurities may include a Michael addition impurity, which can be difficult to remove.

Accordingly, it would be useful to develop new processes and intermediates that can be used to prepare the compound of Formula I. Preferably, these new processes afford final compounds that contain less impurities, such as Michael reaction based impurities, and/or improved M:P atropisomer ratios.

### SUMMARY

Disclosed herein are the compound of Formula I, or pharmaceutically acceptable salts thereof, obtainable by using newly developed intermediates and processes. Also disclosed herein are intermediates useful in preparing the compound of Formula I, or pharmaceutically acceptable salts thereof. Also disclosed herein are methods of making the compound of Formula I, or pharmaceutically acceptable salts thereof.

In an aspect, disclosed herein is a compound of Formula I,

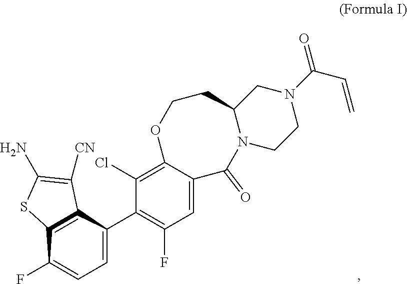

or a pharmaceutically acceptable salt thereof,
obtainable by combining acryloyl chloride and 4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer,

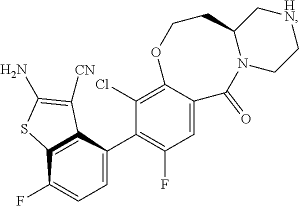

in a continuous stirred-tank reactor, wherein the Michael adduct impurity is less than 1% as measured by HPLC Analysis.

In one aspect, the compound of Formula I, or pharmaceutically acceptable salts thereof, is prepared using the following intermediate

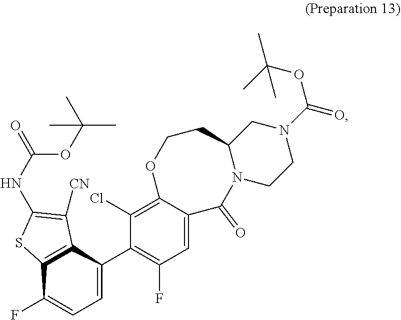

or a pharmaceutically acceptable salt thereof, which is obtainable by reacting tert-butyl (13aS)-9-bromo-10-chloro-8-fluoro-6-oxo-1,3,4,12,13,13a-hexahydropyrazino[2,1-d][1,5]benzoxazocine-2-carboxylate, tert-butyl N-[3-cyano-4-(5,5-dimethyl-1,3,2-dioxaborinan-2-yl)-7-fluoro-benzothiophen-2-yl]carbamate, potassium carbonate, and a catalyst in a solvent, wherein the M:P atropisomer ratio is at least 4:1.

In another aspect, the compound of Formula I, or pharmaceutically acceptable salts thereof, is prepared using the following intermediate,

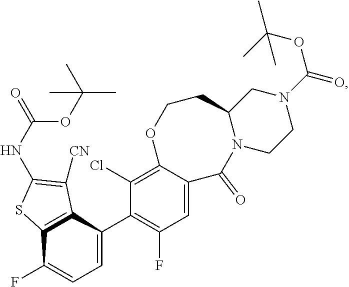

or a pharmaceutically acceptable salt thereof, which is obtainable by reacting tert-butyl (13aS)-9-bromo-10-chloro-8-fluoro-6-oxo-1,3,4,12,13,13a-hexahydropyrazino[2,1-d][1,5]benzoxazocine-2-carboxylate, diacetate[(S)-(−)-2,2′-bis(diphenylphosphino)-1,1′-binaphthyl]palladium(II), tert-butyl N-[3-cyano-4-(5,5-dimethyl-1,3,2-dioxaborinan-2-yl)-7-fluoro-benzothiophen-2-yl]carbamate, abase, and 1,1,1-tris(hydroxymethyl)ethane, wherein the M:P atropisomer ratio of the intermediate is at least 4:1.

Regarding intermediates useful in preparing the compound of Formula I, or pharmaceutically acceptable salts thereof, disclosed herein is an intermediate compound of Formula Ii:

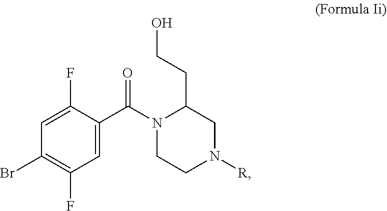

or a pharmaceutically acceptable salt thereof,
wherein:
R is a protecting group.

In an aspect, disclosed herein, the intermediate compound of Formula II is a compound of Formula Iia:

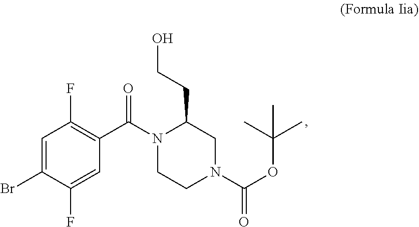 [SMILES: CC(C)(C)OC(=O)N1CCN(C(=O)c2cc(F)c(Br)cc2F)[C@@H](CCO)C1]

or a pharmaceutically acceptable salt thereof.

In an aspect, disclosed herein is a compound of Formula Iia,

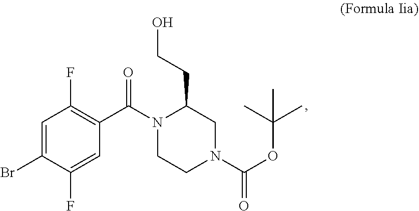 [SMILES: CC(C)(C)OC(=O)N1CCN(C(=O)c2cc(F)c(Br)cc2F)[C@@H](CCO)C1]

or a pharmaceutically acceptable salt thereof,
obtainable by combining tert-butyl (3S)-3-(2-hydroxyethyl)piperazine-1-carboxylate:phosphoric acid (1:1), a base, and 4-bromo-2,5-difluorobenzoic acid, to give tert-butyl (3S)-4-(4-bromo-2,5-difluoro-benzoyl)-3-(2-hydroxyethyl)piperazine-1-carboxylate.

Regarding intermediates useful in preparing the compound of Formula I, or pharmaceutically acceptable salts thereof, disclosed herein is an intermediate compound of Formula IIi:

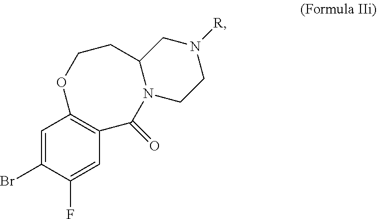

or a pharmaceutically acceptable salt thereof,
wherein:
R is a protecting group.

In an aspect, disclosed herein, the intermediate compound of Formula IIi is a compound of Formula IIia:

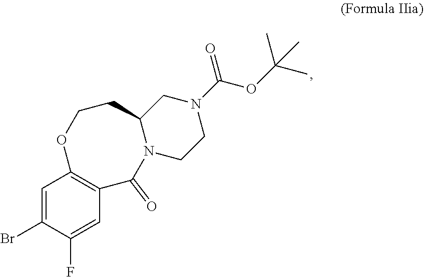 [SMILES: CC(C)(C)OC(=O)N1CCN2C(=O)c3cc(F)c(Br)cc3OCC[C@H]2C1]

or a pharmaceutically acceptable salt thereof.

In an aspect, disclosed herein is an intermediate compound of Formula IIia,

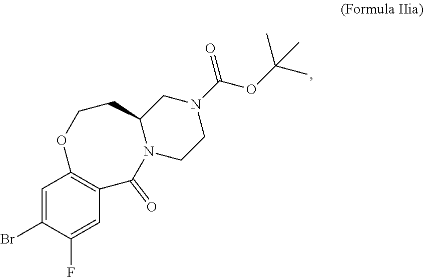 [SMILES: CC(C)(C)OC(=O)N1CCN2C(=O)c3cc(F)c(Br)cc3OCC[C@H]2C1]

or a pharmaceutically acceptable salt thereof,
obtainable by combining tert-butyl (3S)-4-(4-bromo-2,5-difluoro-benzoyl)-3-(2-hydroxyethyl)piperazine-1-carboxylate with a cyclization base to give tert-butyl (13aS)-9-bromo-8-fluoro-6-oxo-1,3,4,12,13,13a-hexahydropyrazino[2,1-d][1,5]benzoxazocine-2-carboxylate.

In an aspect, disclosed herein is an intermediate compound of Formula IIIia,

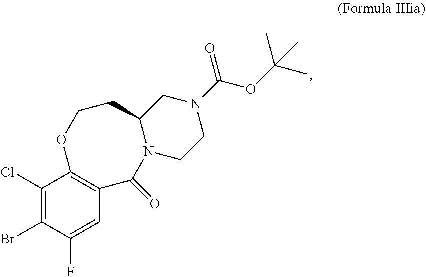 [SMILES: CC(C)(C)OC(=O)N1CCN2C(=O)c3cc(F)c(Br)c(Cl)c3OCC[C@H]2C1]

or a pharmaceutically acceptable salt thereof,
obtainable by combining tert-Butyl (13aS)-9-bromo-8-fluoro-6-oxo-1,3,4,12,13,13a-hexahydropyrazino[2,1-d][1,5]benzoxazocine-2-carboxylate with a chlorinating agent to give tert-Butyl (13aS)-9-bromo-10-chloro-8-fluoro-6-oxo-1,3,4,12,13,13a-hexahydropyrazino[2,1-d][1,5]benzoxazocine-2-carboxylate.

The present disclosure provides a compound selected from the group consisting of:

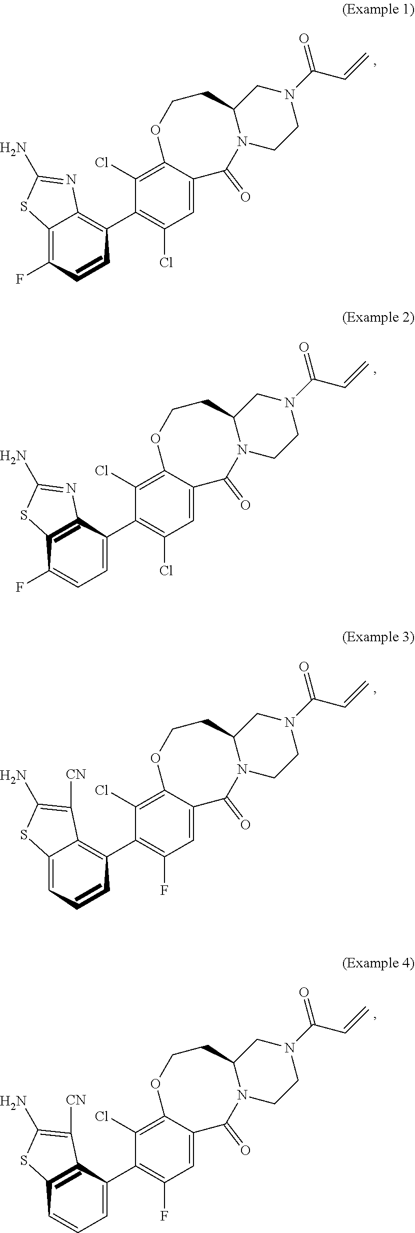

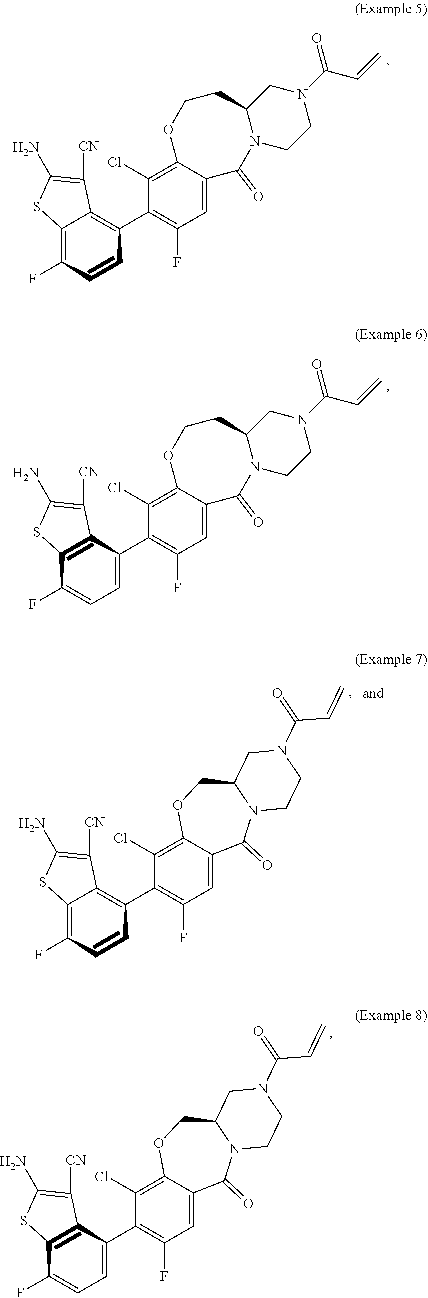

or a pharmaceutically acceptable salt thereof.

The present disclosure also provides the compound selected from the group consisting of:

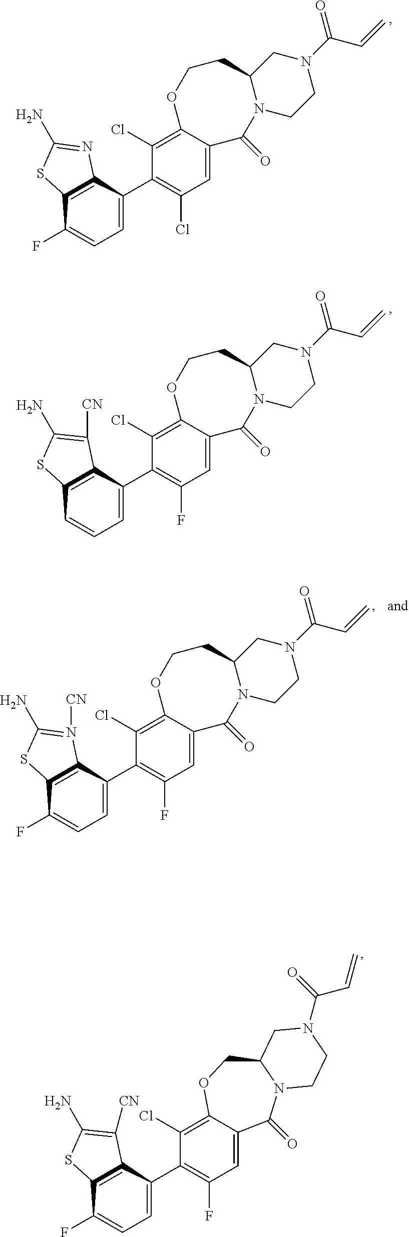

or a pharmaceutically acceptable salt thereof.

The present disclosure also provides the compound selected from the group consisting of:

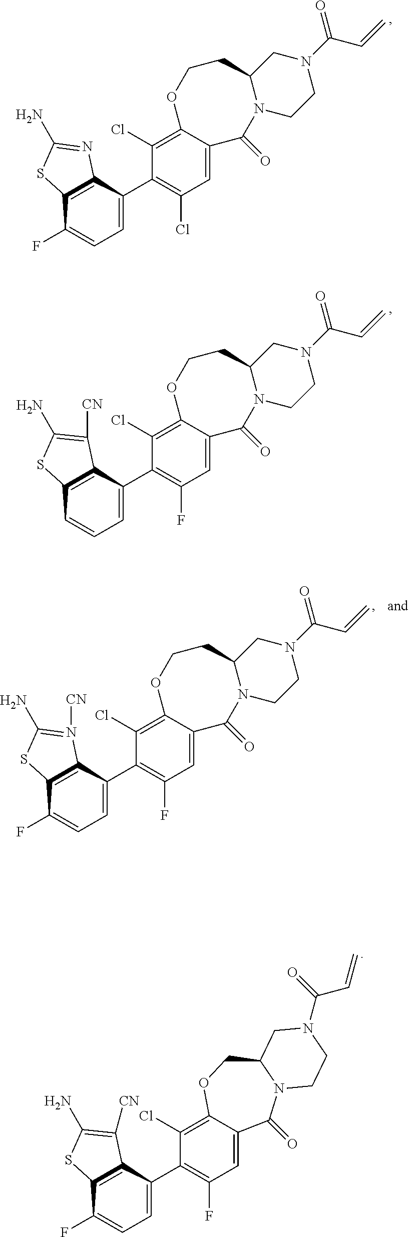

The present disclosure provides the compound selected from the group consisting of:

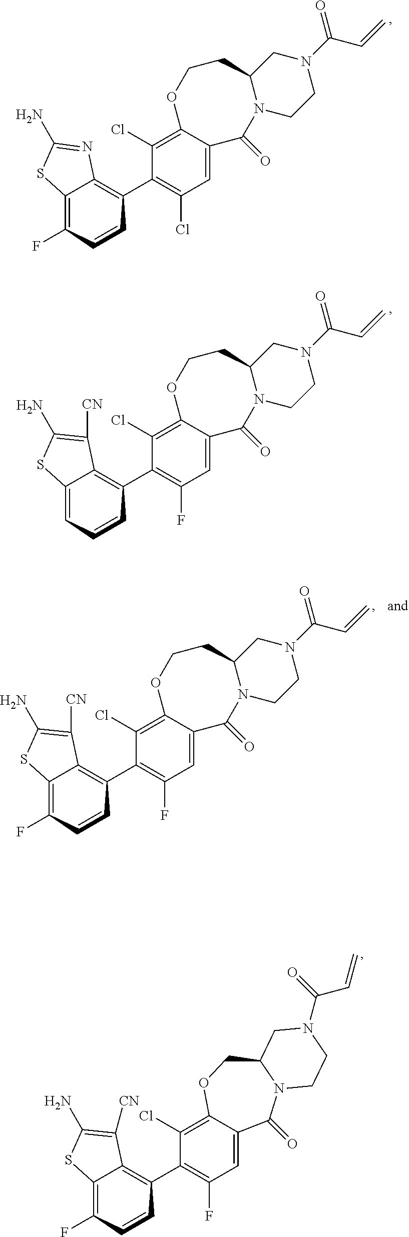

or a pharmaceutically acceptable salt thereof.

The present disclosure provides the compound selected from the group consisting of:

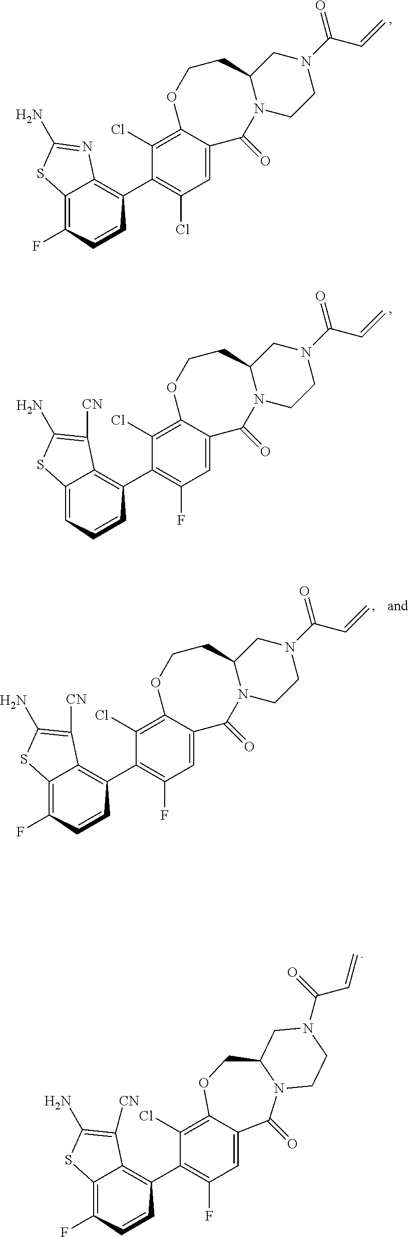

The present disclosure provides the compound selected from the group consisting of:

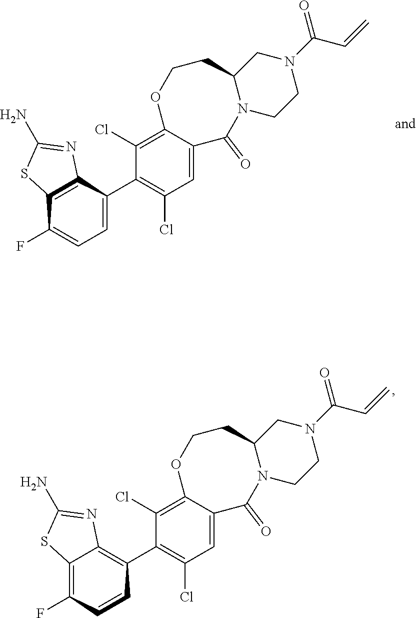

or a pharmaceutically acceptable salt thereof.

The present disclosure provides wherein the compound is

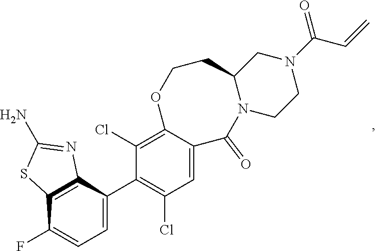

or a pharmaceutically acceptable salt thereof.

The present disclosure provides wherein the compound is

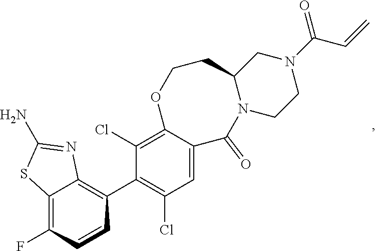

or a pharmaceutically acceptable salt thereof.

The present disclosure provides the compound selected from the group consisting of:

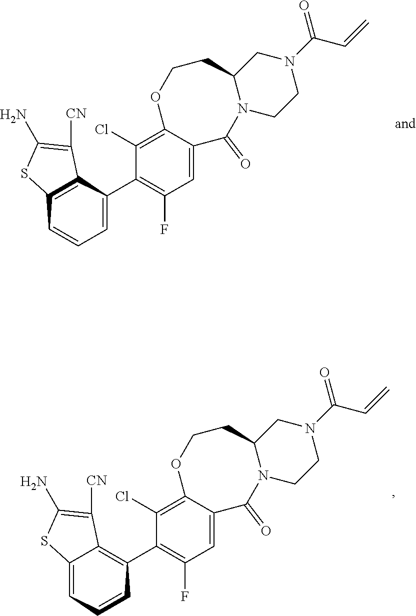

or a pharmaceutically acceptable salt thereof.

The present disclosure provides wherein the compound is

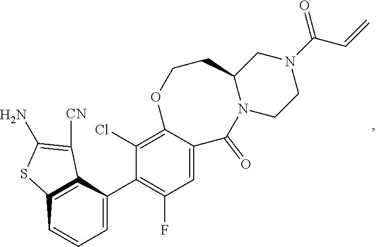

or a pharmaceutically acceptable salt thereof.

The present disclosure provides the compound is

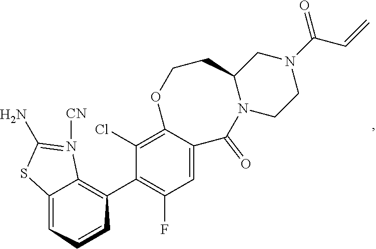

or a pharmaceutically acceptable salt thereof.

The present disclosure provides the compound selected from the group consisting of:

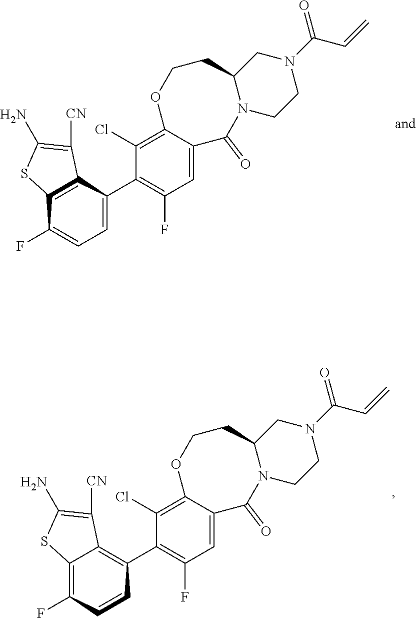

or a pharmaceutically acceptable salt thereof.

The present disclosure provides wherein the compound is

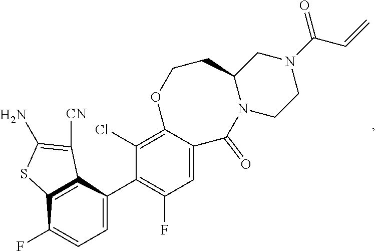

or a pharmaceutically acceptable salt thereof.

The present disclosure provides wherein the compound is

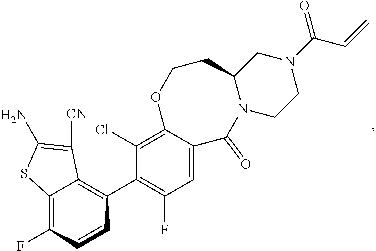

or a pharmaceutically acceptable salt thereof.

The present disclosure provides the compound selected from the group consisting of:

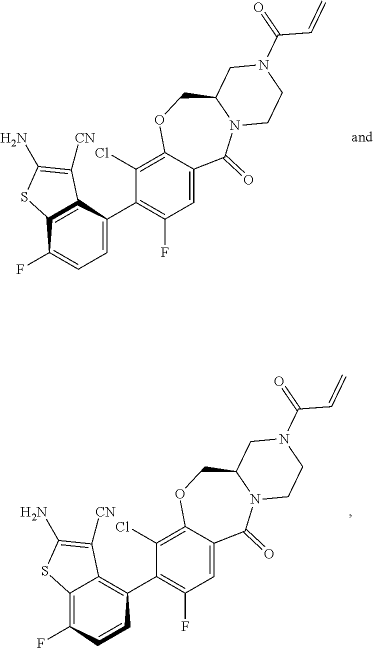

or a pharmaceutically acceptable salt thereof.

The present disclosure provides wherein the compound is

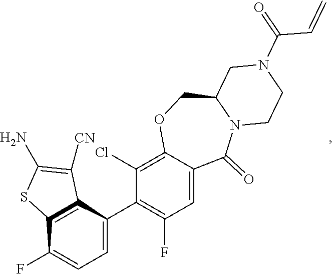

or a pharmaceutically acceptable salt thereof.

The present disclosure provides wherein the compound is

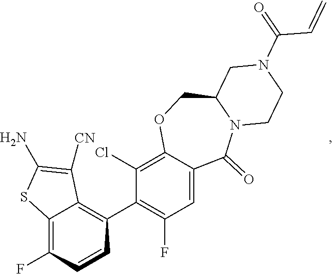

or a pharmaceutically acceptable salt thereof.

The present disclosure also provides a pharmaceutical composition comprising a compound according to any one of Examples 1-8, or a pharmaceutically acceptable salt thereof, and a pharmaceutically acceptable carrier, diluent, or excipient.

The present disclosure also provides a method of treating cancer wherein one or more cells express KRas G12C mutant protein, comprising administering to a patient in need thereof, an effective amount of a compound according to any one of Examples 1-8, or a pharmaceutically acceptable salt thereof. In various embodiments, the cancer is lung cancer, such as advanced non-small cell lung cancer, colorectal cancer, pancreatic cancer, bladder cancer, cervical cancer, endometrial cancer, ovarian cancer, cholangiocarcinoma, or esophageal cancer. In preferred embodiments, the cancer is advanced non-small cell lung cancer, pancreatic cancer, or colorectal cancer. In still more preferred embodiments, the cancer is non-small cell lung cancer.

In still yet another form, the present disclosure comprises a method of treating cancer, comprising administering to a patient in need thereof, an effective amount of a compound according to any one of Examples 1-8, or a pharmaceutically acceptable salt thereof. In another embodiment, the cancer is non-small cell lung carcinoma. In another embodiment, the cancer is colorectal carcinoma. In yet another embodiment, the cancer is mutant pancreatic cancer. In another embodiment, the present disclosure comprising a method of treating KRas G12C mutant bearing cancers of other origins.

The present disclosure also provides a method of treating a patient with a cancer that has a KRAS G12C mutation comprising administering to a patient in need thereof an effective amount of a compound according to any one of Examples 1-8 or a pharmaceutically acceptable salt thereof.

The present disclosure also provides a method of modulating a mutant KRas G12C enzyme in a patient in need thereof, by administering a compound according to any one of Examples 1-8, or a pharmaceutically acceptable salt thereof. Preferably the method comprises inhibiting a human mutant KRas G12C enzyme.

The compounds of the present disclosure, or salts thereof, may be prepared by a variety of procedures, some of which are illustrated in the following Preparations and Examples and by Preparations and Examples of the '877 and '633 references. The compounds of the present disclosure may be prepared by methods well known and appreciated in the art according to the following Preparations and Examples and by Preparations and Examples of the '877 and '633 references. Suitable reaction conditions for the steps of these Preparations and Example are well known in the art and appropriate substitutions of solvents and co-reagents are within the skill of the art. Likewise, it will be appreciated by those skilled in the art that synthetic intermediates may be isolated and/or purified by various well-known techniques as needed or desired, and that frequently, it will be possible to use various intermediates directly in subsequent synthetic steps with little or no purification. As an illustration, compounds of the preparations and examples can be isolated, for example, by silica gel purification, isolated directly by filtration, or crystallization. Furthermore, the skilled artisan will appreciate that in some circumstances, the order in which moieties are introduced is not critical. The particular order of steps required to produce the compounds of the present disclosure is dependent upon the particular compound being synthesized, the starting compound, and the relative liability of the substituted moieties, as is well appreciated by the skilled chemist. All substituents, unless otherwise indicated, are as previously defined, and all reagents are well known and appreciated in the art.

The specific synthetic steps for each of the routes described may be combined in different ways, or in conjunction with steps from different routes, to prepare compounds or salts of the present disclosure. The products of each step in the Preparations below can be recovered by conventional methods, including extraction, evaporation, precipitation, chromatography, filtration, trituration, and crystallization.

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below.

### Definitions

Certain abbreviations are defined as follows: “APCI-MS” refers to atmospheric pressure chemical ionization mass spectrometry; “Boc” refers to tert-butoxycarbonyl; “CDI” refers 1,1′-carbonyldiimidazole; “CDMT” refers to 2-chloro-4,6-dimethoxy-1,3,5-triazine; “DCC” refers to 1,3-dicyclohexylcarbodiimide; “DIC” refers to 1,3-diisopropylcarbodiimide; “DIPEA” refers to N,N-diisopropylethylamine; “DMAc” refers to dimethylacetamide or DMA; “DMAP” refers to 4-dimethylaminopyridine; “DMF” refers to N,N-dimethylformamide; “DMSO” refers to dimethylsulfoxide; “EDCI” refers to 1-(3-dimethylaminopropyl)-3-ethylcarbodiimide hydrochloride; “EtOH” refers to ethanol or ethyl alcohol; “HOAt” refers to 1-hydroxy-7-azobenzotriazole; “HBTU” refers to 3-[bis(dimethylamino)methyliumyl]-3H-benzotriazol-1-oxide hexafluorophosphate; “HOBt” refers to 1-hydroxylbenzotriazole hydrate; “HPLC” refers to high-performance liquid chromatography; “2-MeTHF” refers to 2-methyltetrahydrofuran; “MP (DSC)” refers to melting point by differential scanning calorimetry; “NCS” refers to N-chlorosuccinimide; “NMM” refers to N-methylmorpholine; “NMP” refers to N-methyl-2-pyrrolidone; “NMR” refers to nuclear magnetic resonance; “PG” refers to protecting group; “PyBOP” refers to (benzotriazol-1-yl-oxytripyrrolidinophosphonium hexafluorophosphate); “PyBrOP” refers to bromo(tri-pyrrolidinyl)phosphoniumhexafluorophosphate; and “TEA” refers to triethylamine; “TFA” refers to trifluoroacetic acid; “THF” refers to tetrahydrofuran.

Certain abbreviations are defined as follows: “ACN” refers to acetonitrile; “APCI-MS” refers to atmospheric pressure chemical ionization mass spectrometry; “Boc” refers to tert-butoxycarbonyl; “CDI” refers 1,1′-carbonyldiimidazole; “CDMT” refers to 2-chloro-4,6-dimethoxy-1,3,5-triazine; “DCC” refers to 1,3-dicyclohexylcarbodiimide; “DCM” refers to dichloromethane; “DIC” refers to 1,3-diisopropylcarbodiimide; “DIPEA” or “DIEA” refers to N,N-diisopropylethylamine; “DMAc” or “DMA” refer to dimethylacetamide; “DMAP” refers to 4-dimethylaminopyridine; “DMF” refers to N,N-dimethylformamide; “DMSO” refers to dimethylsulfoxide; “DMSO-d₆” refers to deuterated dimethylsulfoxide; “EDCI” refers to 1-(3-dimethylaminopropyl)-3-ethylcarbodiimide hydrochloride; “EtOAc” refers to ethyl acetate; “EtOH” refers to ethanol or ethyl alcohol; “HATU” refers to 0-(7-Azabenzotriazol-1-yl)-N,N,N′,N′-tetramethyluronium hexafluorophosphate; “HOAt” refers to 1-hydroxy-7-azobenzotriazole; “HBTU” refers to 3-[bis(dimethylamino)methyliumyl]-3H-benzotriazol-1-oxide hexafluorophosphate; “HOBt” refers to 1-hydroxylbenzotriazole hydrate; “HPLC” refers to high-performance liquid chromatography; “MeOH” refers to methanol; “2-MeTHF” refers to 2-methyltetrahydrofuran; “MP (DSC)” refers to melting point by differential scanning calorimetry; “MTBE” refers to methyl tert-butyl ether; “NCS” refers to N-chlorosuccinimide; “NMM” refers to N-methylmorpholine; “NMP” refers to N-methyl-2-pyrrolidone; “NMR” refers to nuclear magnetic resonance; “PG” refers to protecting group; “PyBOP” refers to (benzotriazol-1-yl-oxytripyrrolidinophosphonium hexafluorophosphate); “PyBrOP” refers to bromo(tri-pyrrolidinyl)phosphoniumhexafluorophosphate; and “TEA” refers to triethylamine; “TFA” refers to trifluoroacetic acid; and “THF” refers to tetrahydrofuran.

A “base” is a molecule that is a proton acceptor, or a molecule that can neutralize an acid. Examples of bases include but are not limited to, K₂CO₃.

A “cyclization base” is a base which assists in ring formation. Examples of cyclization bases include sodium hydride, N,N-diisopropylethylamine (DIPEA or DIEA), triethylamine (TEA), cesium carbonate, diazabicycloundecene (DBU), sodium tert-butoxide, sodium tert-pentoxide, sodium tert-amylate, potassium tert-pentoxide, or potassium tert-butoxide.

A “cyclization solvent” is a solvent which assists in cyclization reaction. Examples of cyclization solvents include DMF, NMP, DMAc, DMSO, or THF.

A “chlorinating agent” is an agent that assists in chlorinating a molecule. Examples of chlorinating agents include trichloroisocyanuric acid or NCS.

The terms “continuous manufacturing” (herein after “CM”), “flow”, and “flow chemistry” involve the continuous feeding of input materials into, the transformation of in-process materials within, and the concomitant removal of output materials from a manufacturing process, such as a continuous stirred-tank reactor. The terms are applicable to CM for new products (e.g., new drugs, generic drugs, biosimilars) and the conversion of batch manufacturing to CM for existing products. (See for example, FDA, “Q13 Continuous Manufacturing of Drug Substances and Drug Products, Guidance for Industry”, March 2023).

The terms “batch”, “batch manufacturing” and “batch chemistry” involve a specific quantity of material produced in a process or series of processes so that it is expected to be homogeneous within specified limits. The batch size can be defined either by a fixed quantity or by the amount produced in a fixed time interval. (See for example, FDA, “Q7 Good Manufacturing Practice Guidance for Active Pharmaceutical Ingredients, Guidance for Industry”, September 2016).

A “catalyst” is a substance that increases the rate of a chemical reaction without itself undergoing any permanent chemical change.

The term “M:P atropisomer ratio” is the atropisomer ratio between corresponding M and P atropisomers.

A “protecting group” is a reversibly formed derivative of an existing functional group in a molecule. Several classes of protecting groups include alcohol protecting groups, amine protecting groups, carbonyl protecting groups, carboxylic acid protecting groups, phosphate protecting groups, and terminal alkyne protecting groups. Carboxylic acid protecting groups include types of protecting groups such as tert-butyl esters.

As used herein, the terms “treating”, “to treat”, or “treatment”, includes slowing, reducing, or reversing the progression or severity of an existing symptom, disorder, condition, which can include specifically slowing the growth of a cancerous lesion or progression of abnormal cell growth and/or cell division.

The term “PD-1” as used herein refers to programmed death receptor 1. The term “PD-L1” as used herein refers to programmed death ligand 1.

The term “CDK4/CDK6 inhibitor” as used herein refers to any chemical that inhibits the function of CDK4/CDK6.

The term “EGFR inhibitor” as used herein refers to any chemical that inhibits the function of EGFR.

The term “ERK” refers to extracellular signal-regulated kinases. The term “ERK inhibitor” as used herein refers to any chemical that inhibits the function of ERK.

The term “platinum agent” as used herein refers to any platinum containing chemical that inhibits cancer.

The term “antifolate” as used herein refers to any chemical that inhibits the function of folic acid. In an embodiment, the antifolate is pemetrexed.

The term “Aurora A inhibitor” as used herein refers to any chemical that inhibits the function of Aurora A kinase.

The term “SHP2 inhibitor” as used herein refers to any chemical that inhibits the function of SHP2.

In another aspect, disclosed herein is a method of treating a KRAS G12C mutant cancer comprising: administering to a patient in need thereof, a dose between about 50 mg and about 200 mg of a compound of Formula I, or a pharmaceutically acceptable salt thereof, in simultaneous, separate or sequential combination with a PD-1 or PD-L1 inhibitor, for use in the treatment of a KRAS G12C mutant cancer. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with pembrolizumab in the treatment of KRAS G12C-mutant advanced NSCLC. In another embodiment, the PD-1 or PD-L1 inhibitor is pembrolizumab, wherein pembrolizumab is dosed at 200 mg once every three weeks. In another embodiment, the PD-1 or PD-L1 inhibitor is nivolumab. In another embodiment, the PD-1 or PD-L1 inhibitor is cimiplimab. In another embodiment, the PD-1 or PD-L1 inhibitor is sintilimab. In another embodiment, the PD-1 or PD-L1 inhibitor is atezolizumab. In another embodiment, the PD-1 or PD-L1 inhibitor is avelumab. In another embodiment, the PD-1 or PD-L1 inhibitor is durvalumab. In another embodiment, the PD-1 or PD-L1 inhibitor is lodapilimab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with pembrolizumab in the treatment of KRAS G12C-mutant advanced NSCLC, wherein pembrolizumab is dosed intravenously at 200 mg once every three weeks. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with a CDK4/CDK6 inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with abemaciclib in the treatment of KRAS G12C-mutant advanced NSCLC. In another embodiment, the CDK4/CDK6 inhibitor is abemaciclib, wherein abemaciclib is dosed at 150 mg BID. In another embodiment, the CDK4/CDK6 inhibitor is palbociclib. In another embodiment, the CDK4/CDK6 inhibitor is ribociclib. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with abemaciclib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with an EGFR inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with erlotinib in the treatment of KRAS G12C-mutant advanced NSCLC. In another embodiment, the EGFR inhibitor is erlotinib, wherein erlotinib is dosed at 150 mg once a day. In another embodiment, the EGFR inhibitor is afatinib. In another embodiment, the EGFR inhibitor is gefitinib. In another embodiment, the EGFR inhibitor is cetuximab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with erlotinib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with cetuximab in the treatment of KRAS G12C-mutant advanced CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with an ERK inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with LY3214996 in the treatment of KRAS G12C-mutant advanced NSCLC or CRC. In another embodiment, the ERK inhibitor is LY3214996, wherein LY3214996 is dosed at 400 mg twice a day. In another embodiment, the ERK inhibitor is LTT462. In another embodiment, the ERK inhibitor is K₀-947. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with LY3214996 in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with a platinum agent. In another embodiment, the platinum agent is cisplatin. In another embodiment, the platinum agent is carboplatin. In another embodiment, the platinum agent is oxaliplatin.

In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, for use in simultaneous, separate or sequential combination with an antifolate, for the treatment of a KRAS G12C mutant cancer. In another embodiment, the antifolate is pemetrexed. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and an Aurora A inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid: 2-methylpropan-2-amine (1:1) salt in the treatment of KRAS G12C-mutant advanced NSCLC. In another embodiment, the Aurora A inhibitor is an aminopyridine compound, or a pharmaceutically acceptable salt thereof. In another embodiment, the Aurora A inhibitor is an Aurora A selective inhibitor, or a pharmaceutically acceptable salt thereof. In another embodiment, the Aurora A inhibitor is alisertib as described in WO 2008/063525. In another embodiment, the Aurora A inhibitor is a pan Aurora inhibitor, or a pharmaceutically acceptable salt thereof. In another embodiment, the Aurora A inhibitor is tozasertib as described in WO 2004/000833. In another embodiment, the Aurora A inhibitor is danusertib as described in WO 2005/005427. In another embodiment, the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:

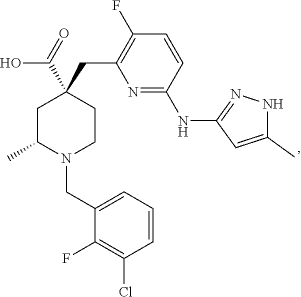

or a pharmaceutically acceptable salt thereof. In an embodiment, the pharmaceutically acceptable salt is an amine salt. One example of the amine salt is NH₃ amine salt. Another example of the amine salt is 2-methylpropan-2-amine salt.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, which has the following structure:

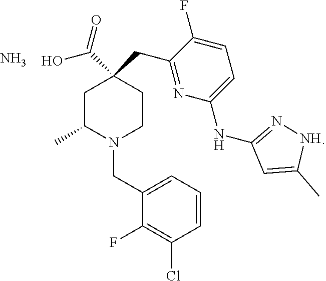

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid: 2-methylpropan-2-amine (1:1) salt, which has the following structure:

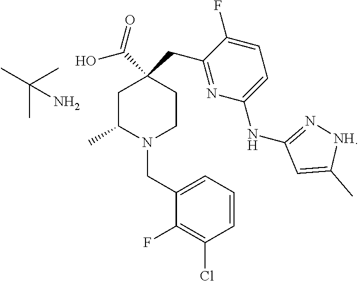

In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of a compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid: 2-methylpropan-2-amine (1:1) salt in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and the SHP2 inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with TNO155 in the treatment of KRAS G12C-mutant advanced NSCLC or CRC. In an embodiment, the SHP2 inhibitor is a Type I SHP2 Inhibitor or a Type II SHP2 Inhibitor. In another embodiment, the Type I SHP2 inhibitor is PHPS1 or GS-493, or a pharmaceutically acceptable salt thereof. In another embodiment, the Type I SHP2 inhibitor is NSC-87877 or NSC-117199, or a pharmaceutically acceptable salt thereof. In another embodiment, the Type I SHP2 inhibitor is cefsulodin, or a pharmaceutically acceptable salt thereof. In another embodiment, the Type II SHP2 inhibitor is JAB-3068 or JAB-3312, or a pharmaceutically acceptable salt thereof. In another embodiment, the Type II SHP2 inhibitor is RMC-4550 or RMC-4630, or a pharmaceutically acceptable salt thereof. In another embodiment, the Type II SHP2 inhibitor is a SHP099, SHP244, SHP389, SHP394, or TNO155, or a pharmaceutically acceptable salt thereof. In another embodiment, the Type II SHP2 inhibitor is RG-6433 or RLY-1971, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and RMC-4630. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and JAB-3068. In another embodiment, the SHP2 inhibitor is BBP-398, IACS-15509, or IACS-13909, or a pharmaceutically acceptable salt thereof. In another embodiment, the SHP2 inhibitor is X37, or a pharmaceutically acceptable salt thereof. In another embodiment, the SHP2 inhibitor is ERAS-601, or a pharmaceutically acceptable salt thereof. In another embodiment, the SHP2 inhibitor is SH₃₈₀₉, or a pharmaceutically acceptable salt thereof. In another embodiment, the SHP2 inhibitor is HBI-2376, or a pharmaceutically acceptable salt thereof. In another embodiment, the SHP2 inhibitor is ETS-001, or a pharmaceutically acceptable salt thereof. In another embodiment, the SHP2 inhibitor is PCC0208023, or a pharmaceutically acceptable salt thereof. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a method of treating a KRAS G12C mutant cancer, comprising: administering to a patient in need thereof, a dose between about 50 mg and about 200 mg of a compound of Formula I, or a pharmaceutically acceptable salt thereof, in simultaneous, separate or sequential combination with TNO155 in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a use of a compound of Formula I, or a pharmaceutically acceptable salt thereof, administering to a patient in need thereof, a dose between about 50 mg and about 200 mg of the compound of Formula I, or a pharmaceutically acceptable salt thereof, in simultaneous, separate or sequential combination with a second therapeutic agent.

In another aspect, disclosed herein is a use of a compound of Formula I, or a pharmaceutically acceptable salt thereof, in the manufacture of a medicament for treating a KRAS G12C mutant cancer, wherein the compound is administered at a dose between about 50 mg and about 200 mg, in simultaneous, separate or sequential combination with a second therapeutic agent, wherein the second therapeutic agent is selected from the group consisting of: one or more of a PD-1 inhibitor, or a pharmaceutically acceptable salt thereof, a PD-L1 inhibitor, or a pharmaceutically acceptable salt thereof, a CDK4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, or a pharmaceutically acceptable salt thereof, an antifolate, or a pharmaceutically acceptable salt thereof, an Aurora A inhibitor, or a pharmaceutically acceptable salt thereof, and a SHP2 inhibitor, or pharmaceutically acceptable salts thereof.

In another aspect, disclosed herein is a use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with a PD-1 or PD-L1 inhibitor, for treatment of a KRAS G12C mutant cancer. In another embodiment, the PD-1 or PD-L1 inhibitor is pembrolizumab, wherein pembrolizumab is dosed at 200 mg once every three weeks. In another embodiment, the PD-1 or PD-L1 inhibitor is nivolumab. In another embodiment, the PD-1 or PD-L1 inhibitor is cimiplimab. In another embodiment, the PD-1 or PD-L1 inhibitor is sintilimab. In another embodiment, the PD-1 or PD-L1 inhibitor is atezolizumab. In another embodiment, the PD-1 or PD-L1 inhibitor is avelumab. In another embodiment, the PD-1 or PD-L1 inhibitor is durvalumab. In another embodiment, the PD-1 or PD-L1 inhibitor is lodapilimab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is a use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with pembrolizumab in the treatment of KRAS G12C-mutant advanced NSCLC, wherein pembrolizumab is dosed intravenously at 200 mg once every three weeks. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg. In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with a CDK4/CDK6 inhibitor, or a pharmaceutically acceptable salt thereof. In another embodiment, the CDK4/CDK6 inhibitor is abemaciclib, wherein abemaciclib is dosed at 150 mg BID. In another embodiment, the CDK4/CDK6 inhibitor is palbociclib. In another embodiment, the CDK4/CDK6 inhibitor is ribociclib. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with abemaciclib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with an EGFR inhibitor, or a pharmaceutically acceptable salt thereof. In another embodiment, the EGFR inhibitor is erlotinib, wherein erlotinib is dosed at 150 mg once a day. In another embodiment, the EGFR inhibitor is afatinib. In another embodiment, the EGFR inhibitor is gefitinib. In another embodiment, the EGFR inhibitor is cetuximab. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with erlotinib in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with cetuximab in the treatment of KRAS G12C-mutant advanced CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with an ERK inhibitor, or a pharmaceutically acceptable salt thereof. In another embodiment, the ERK inhibitor is LY3214996, wherein LY3214996 is dosed at 400 mg twice a day. In another embodiment, the ERK inhibitor is LTT462 In another embodiment, the ERK inhibitor is KO-947. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with LY3214996 in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with a platinum agent. In another embodiment, the platinum agent is cisplatin. In another embodiment, the platinum agent is carboplatin. In another embodiment, the platinum agent is oxaliplatin. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, for use in simultaneous, separate or sequential combination with an antifolate, for the treatment of a KRAS G12C mutant cancer. In another embodiment, the antifolate is pemetrexed. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, and an Aurora A inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid: 2-methylpropan-2-amine (1:1) salt in the treatment of KRAS G12C-mutant advanced NSCLC. In another embodiment, the Aurora A inhibitor is an aminopyridine compound, or a pharmaceutically acceptable salt thereof. In another embodiment, the Aurora A inhibitor is an Aurora A selective inhibitor, or a pharmaceutically acceptable salt thereof. In another embodiment, the Aurora A inhibitor is alisertib as described in WO 2008/063525. In another embodiment, the Aurora A inhibitor is a pan Aurora inhibitor, or a pharmaceutically acceptable salt thereof. In another embodiment, the Aurora A inhibitor is tozasertib as described in WO 2004/000833. In another embodiment, the Aurora A inhibitor is danusertib as described in WO 2005/005427. In another embodiment, the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with (2R,4R)-1-[(3-chloro-2-fluoro-phenyl)methyl]-4-[[3-fluoro-6-[(5-methyl-1h-pyrazol-3-yl)amino]-2-pyridyl]methyl]-2-methyl-piperidine-4-carboxylic acid: 2-methylpropan-2-amine (1:1) salt in the treatment of KRAS G12C-mutant advanced NSCLC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, and the SHP2 inhibitor, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, in simultaneous, separate or sequential combination with TNO155 in the treatment of KRAS G12C-mutant advanced NSCLC or CRC. In an embodiment, the SHP2 inhibitor is a Type I SHP2 Inhibitor or a Type II SHP2 Inhibitor. In another embodiment, the Type I SHP2 inhibitor is PHPS1 or GS-493, or a pharmaceutically acceptable salt thereof. In another embodiment, the Type I SHP2 inhibitor is NSC-87877 or NSC-117199, or a pharmaceutically acceptable salt thereof. In another embodiment, the Type I SHP2 inhibitor is cefsulodin, or a pharmaceutically acceptable salt thereof. In another embodiment, the Type II SHP2 inhibitor is JAB-3068 or JAB-3312, or a pharmaceutically acceptable salt thereof. In another embodiment, the Type II SHP2 inhibitor is RMC-4550 or RMC-4630, or a pharmaceutically acceptable salt thereof. In another embodiment, the Type II SHP2 inhibitor is a SHP099, SHP244, SHP389, SHP394, or TNO155, or a pharmaceutically acceptable salt thereof. In another embodiment, the Type II SHP2 inhibitor is RG-6433 or RLY-1971, or a pharmaceutically acceptable salt thereof. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and RMC-4630. In another aspect, disclosed herein is a method of treating a KRAS G12C mutant cancer, comprising administering to a patient in need thereof, a dose between about 50 mg and about 200 mg of a compound of Formula I, or a pharmaceutically acceptable salt thereof, and JAB-3068. In another embodiment, the SHP2 inhibitor is BBP-398, IACS-15509, or IACS-13909, or a pharmaceutically acceptable salt thereof. In another embodiment, the SHP2 inhibitor is X37, or a pharmaceutically acceptable salt thereof. In another embodiment, the SHP2 inhibitor is ERAS-601, or a pharmaceutically acceptable salt thereof. In another embodiment, the SHP2 inhibitor is SH₃₈₀₉, or a pharmaceutically acceptable salt thereof. In another embodiment, the SHP2 inhibitor is HBI-2376, or a pharmaceutically acceptable salt thereof. In another embodiment, the SHP2 inhibitor is ETS-001, or a pharmaceutically acceptable salt thereof. In another embodiment, the SHP2 inhibitor is PCC0208023, or a pharmaceutically acceptable salt thereof. In an embodiment, the dose of the compound of Formula I, or a pharmaceutically acceptable salt thereof, is selected from the group consisting of about 50 mg, about 100 mg, about 150 mg, and about 200 mg.

In another aspect, disclosed herein is use of a compound of Formula I, or a pharmaceutically acceptable salt thereof, in the manufacture of a medicament, administering to a patient in need thereof, a dose between about 50 mg and about 200 mg of a compound of Formula I, or a pharmaceutically acceptable salt thereof, in simultaneous, separate or sequential combination with TNO155 in the treatment of KRAS G12C-mutant advanced NSCLC or CRC. In an embodiment, the dose of the compound of Formula I, or a pharmaceutically acceptable salt thereof, is administered once a day. In an embodiment, the dose of the compound of Formula I, or a pharmaceutically acceptable salt thereof, is administered twice a day. In an embodiment, the dose of the compound of Formula I, or a pharmaceutically acceptable salt thereof, is selected from the group consisting of about 50 mg, about 100 mg, about 150 mg, and about 200 mg.

Compounds of the present disclosure can be synthesized in part by following the steps outlined in the following Schemes 1-1a which comprise different sequences of assembling intermediates or compounds. Starting materials are either commercially available or made by known procedures in the reported literature or as illustrated below.

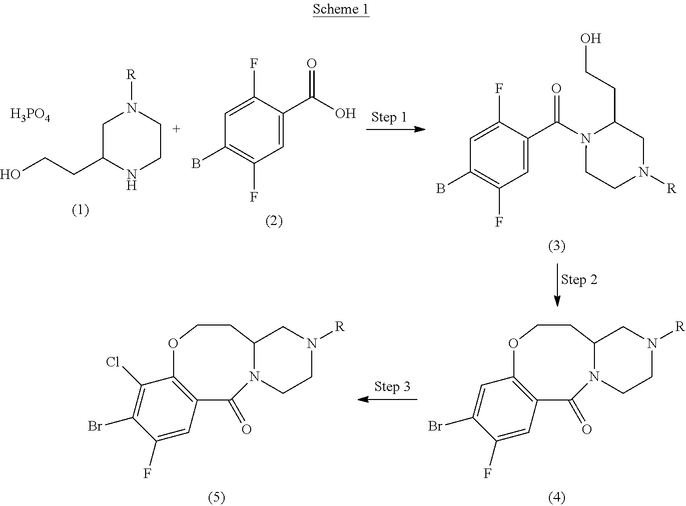

In Step 1 of Scheme 1, the protected piperazine-2-ethanol, compound (1), is coupled with 4-bromo-2,5-difluoro-benzoic acid, compound (2), in an amide bond formation using a coupling reagent such as CDMT with an organic base such as NMM in a solvent system such as acetonitrile and water and an inorganic base such as K₂CO₃. R is a protecting group developed for the amino group, such as carbamates and amides.

Such protecting groups are well known and appreciated in the art, such as carbamate protecting groups including allyloxycarbonyl, fluorenylmethoxycarbonyl, or benzyloxycarbonyl. A common and preferred protecting group can be Boc. One skilled in the art will recognize that there are a number of methods and reagents for amide formation resulting from the reaction of carboxylic acids and amines. For example, the reaction of the amine compound with an appropriate carboxylic acid in the presence of a coupling reagent with or without an organic base such as DIPEA or TEA can provide compound (3). Other coupling reagents include carbodiimides, such as DCC, DIC, EDCI or a carbonyldiimidazole such as CDI. Amide coupling additives, such as HOBt and HOAt can also be used to enhance the reaction. Additionally, uronium or phosphonium salts of non-nucleophilic anions, such as HBTU, HATU, PyBOP, and PyBrOP could be used in place of the more traditional coupling reagents. An additive such as DMAP may be used to enhance the reaction. Alternatively, the acid chloride of compound (2) can be used in the presence of a base, such as TEA or pyridine to give compound (3).

In Step 2, the intramolecular cyclization of compound (3) is completed using an appropriate base such as potassium tert-butoxide, sodium tert-amylate, sodium tert-butoxide, sodium tert-pentoxide, DIPEA, TEA, DBU, sodium hydride in a solvent such as DMF to give compound 4. Other possible solvents could be NMP, DMAc, DMSO, and THF. This intramolecular cyclization of compound (3) to compound (4) may be conducted by slowly adding a solution of compound (3) to an excess of base so as to minimize intermolecular reaction derived impurities.

In Step 3, compound (4) can be chlorinated with under acidic conditions using an acid such as TFA, with a chlorinating agent such as trichloroisocyanuric acid or NCS in a solvent such as acetonitrile or DMF to give compound (5).

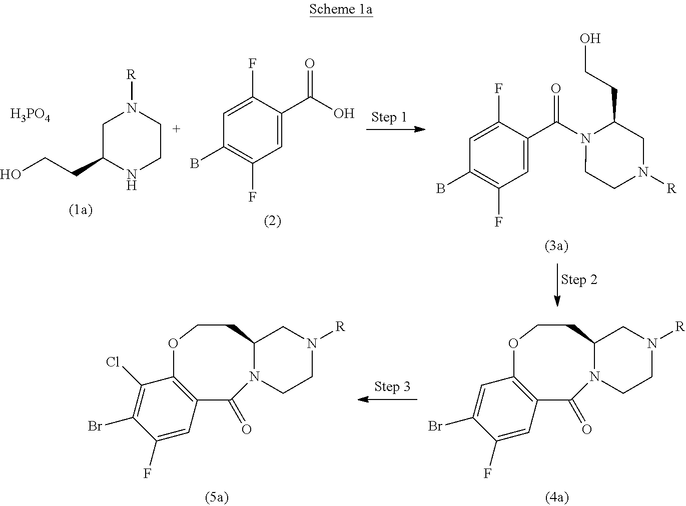

Scheme 1a illustrates a chiral synthesis of compound (5a). Compound (1a) can be prepared as described by Medicinal Chemistry route to 1, Development of an Alternative Route to the Bicyclic Piperazine, Retrosynthetic analysis of bicyclic piperazine core 2, and/or Coupling, cyclization, reduction, and Michael addition to afford Piperazine 24 in Org Proc Res Dev., 2011, 15(6). 1328-1335. Compounds (3a), (4a), and (5a) can be prepared as described in Scheme 1. tert-Butyl (S)-9-bromo-10-fluoro-12-oxo-1,2,4,4a,5,6-hexahydro-3H,12H-benzo[b]pyrazino[1,2-e][1,5]oxazocine-3-carboxylate is synthesized using a 7-step sequence beginning with commercially available S-aspartic acid, which is the source of the stereocenter.

It is known that significant impurity rejection including rejection of the R-enantiomer can be achieved in the isolation of intermediates. tert-Butyl (S)-9-bromo-10-fluoro-12-oxo-1,2,4,4a,5,6-hexahydro-3H,12H-benzo[b]pyrazino[1,2-e][1,5]oxazocine-3-carboxylate can be isolated by crystallization with significant impurity rejection. Steps and Schemes are used to control process and reagent impurities.

The present disclosure provides a method of preparation of an intermediate compound of the Formula IIa:

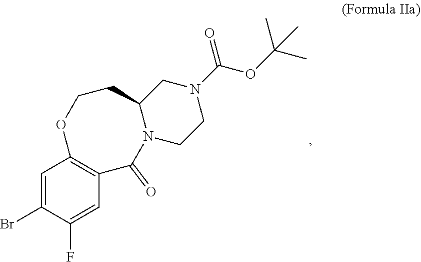 [SMILES: CC(C)(C)OC(=O)N1CCN2C(=O)c3cc(F)c(Br)cc3OCC[C@H]2C1]

or a pharmaceutically acceptable salt thereof, comprising:
cyclization of an intermediate compound of the Formula I:

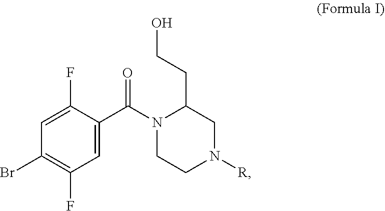

### or of the Formula Ia:

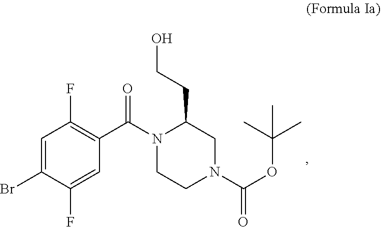 [SMILES: CC(C)(C)OC(=O)N1CCN(C(=O)c2cc(F)c(Br)cc2F)[C@@H](CCO)C1]

or a pharmaceutically acceptable salt thereof, by use of a cyclization base.

In an embodiment, the cyclization base is selected from the group consisting of sodium hydride, N,N-diisopropylethylamine (DIPEA or DIEA), triethylamine (TEA), cesium carbonate, diazabicycloundecene (DBU), sodium tert-butoxide, sodium tert-pentoxide, sodium tert-amylate, potassium tert-pentoxide, and potassium tert-butoxide.

In another embodiment, the method of preparation further comprises a cyclization solvent. In an embodiment the cyclization solvent is N,N-dimethylformamide (DMF).

The method of preparation wherein the step of cyclization is conducted at about 0° C.

### DETAILED DESCRIPTION

A KRAS G12C inhibitor of interest is 4-[(13aS)-10-chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, which has the following structure:

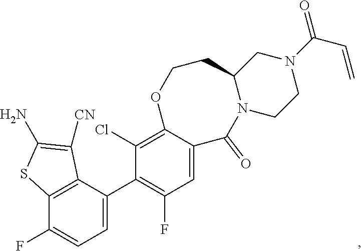 [SMILES: C=CC(=O)N1CCN2C(=O)c3cc(F)c(-c4ccc(F)c5sc(N)c(C#N)c45)c(Cl)c3OCC[C@H]2C1]

This compound was disclosed as Example 35 in each of the '877 and '633 references. As disclosed therein, this compound exists as atropisomers. The atropisomers may be separated. See Preparations 167 and 168 of the '877 and '633 references. The desired diastereomer is taught in Preparation 167 of the '877 and '633 references.

This desired diastereomer corresponds to an M atropisomer. The second diastereomer may be further processed to 4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer, which has the following structure:

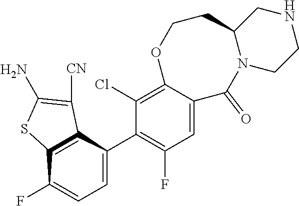

4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer in each of the '877 and '633 references may be further processed to Example 35, M atropisomer in each of the '877 and '633 references.

Example 35, M atropisomer is 4-[(13aS)-10-chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, M atropisomer (hereinafter “Formula I”) having the following structure:

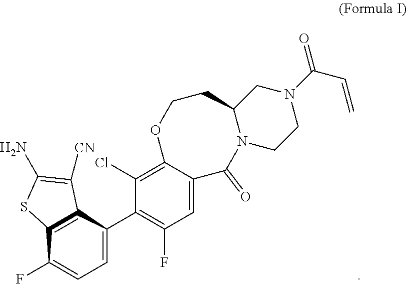

The compound of Formula I is currently undergoing clinical testing (ClinicalTrials.gov Identifier: NCT04956640) to assess its utility in treating patients having cancer that is treatable by inhibiting KRAS G12C. The compound of Formula I may be used, either as a monotherapy, in combination with one or more other therapeutic agents, or as part of neoadjuvant, adjuvant, advanced, or metastatic therapy, to treat cancer. Such cancers include but are not limited to lung cancer, colorectal cancer, pancreatic cancer, bladder cancer, cervical cancer, endometrial cancer, ovarian cancer, cholangiocarcinoma or esophageal cancer.

Another example of a procedure of interest is Scheme 18 disclosed in each of the '877 and '633 references. As disclosed therein, Scheme 18 shows a coupling of compound 37 and compound 87 and subsequent cyclization to give compound 88.

Another example of a procedure of interest is Step D of Scheme 20 disclosed in each of the '877 and '633 references. As disclosed therein, Step D shows a coupling between compound (97) and a partner to give compounds of Formula I. The partner can be an acid chloride, a carboxylic acid, or cyanogen bromide. In cases with an acid chloride, a suitable base such as TEA or DIEA is used in a solvent such as DCM. The acid chloride can also be used with potassium carbonate as the base in a biphasic solvent system such as EtOAc, THF, and water. In cases where the partner is a carboxylic acid, the conditions include a suitable coupling reagent such as propylphosphonic anhydride with a suitable base such as DIEA in a solvent such as DMF. If the partner is cyanogen bromide, a suitable base such as aqueous NaOH is utilized in a solvent such as DCM.

The compounds of the present disclosure, or salts thereof, may be prepared by a variety of procedures, some of which are illustrated in the Preparations and Examples below. The specific synthetic steps for each of the routes described may be combined in different ways, or in conjunction with steps from different routes, to prepare compounds or salts of the present disclosure. The products of each step in the Preparations below can be recovered by conventional methods, including extraction, evaporation, precipitation, chromatography, filtration, trituration, and crystallization.

The compounds of the present disclosure may be prepared according to the following Preparations and Example by methods well known and appreciated in the art. Suitable reaction conditions for the steps of these Preparations and Example are well known in the art and appropriate substitutions of solvents and co-reagents are within the skill of the art. Likewise, it will be appreciated by those skilled in the art that synthetic intermediates may be isolated and/or purified by various well known techniques as needed or desired, and that frequently, it will be possible to use various intermediates directly in subsequent synthetic steps with little or no purification.

By way of example, compounds of the present disclosure can be synthesized using the methods described below, together with synthetic methods known in the art of synthetic organic chemistry, or variations thereon as appreciated by those skilled in the art. As an illustration, compounds of the preparations and examples can be isolated, for example, by silica gel purification, isolated directly by filtration, or crystallization. Furthermore, the skilled artisan will appreciate that in some circumstances, the order in which moieties are introduced is not critical. The particular order of steps required to produce the compounds of the present disclosure is dependent upon the particular compound being synthesized, the starting compound, and the relative liability of the substituted moieties, as is well appreciated by the skilled chemist. All substituents, unless otherwise indicated, are as previously defined, and all reagents are well known and appreciated in the art.

Also disclosed herein is a method of preparation of an intermediate compound of Formula Iia, or a pharmaceutically acceptable salt thereof, comprising combining tert-butyl (3S)-3-(2-hydroxyethyl)piperazine-1-carboxylate:phosphoric acid (1:1), a base, and 4-bromo-2,5-difluorobenzoic acid,

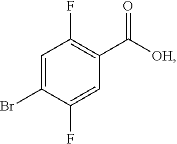 [SMILES: O=C(O)c1cc(F)c(Br)cc1F]

to give tert-butyl (3S)-4-(4-bromo-2,5-difluoro-benzoyl)-3-(2-hydroxyethyl)piperazine-1-carboxylate. In an embodiment, the step of combining further comprises the steps of adding CDMT, acetonitrile, and NMM to the 4-bromo-2,5-difluorobenzoic acid. In another embodiment, the step of combining further comprises the steps of adding K₂CO₃ and water to the tert-butyl (3S)-3-(2-hydroxyethyl)piperazine-1-carboxylate:phosphoric acid (1:1).

Also disclosed herein is a method of preparation of an intermediate compound of Formula Ilia, or a pharmaceutically acceptable salt thereof, comprising cyclization of an intermediate compound of Formula II, or a pharmaceutically acceptable salt thereof, or Formula Iia, or a pharmaceutically acceptable salt thereof, by use of a cyclization base. In an embodiment, the cyclization base is selected from the group consisting of sodium hydride, N,N-diisopropylethylamine (DIPEA), triethylamine (TEA), cesium carbonate, diazabicycloundecene (DBU), sodium tert-butoxide, sodium tert-pentoxide, sodium tert-amylate, potassium tert-pentoxide, and potassium tert-butoxide. In another embodiment, the method of preparation further comprises a cyclization solvent. In yet another embodiment, the cyclization solvent is N,N-dimethylformamide (DMF). In yet another embodiment, the method of preparation wherein the step of cyclization is conducted at about 0° C.

Also disclosed herein is a method of preparation of a compound of Formula I comprising combining acryloyl chloride and 4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer in a continuous stirred-tank reactor. In an embodiment, the step of combining further comprises adding a feed A including phosphate and water. In an embodiment, the phosphate includes monopotassium phosphate and dipotassium phosphate. In yet another embodiment, the acryloyl chloride is included in a Feed B, wherein the Feed B includes 2-methyltetrahydrofuran. In yet another embodiment, the 4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer is included in a Feed C, wherein the Feed C includes 2-methyltetrahydrofuran and water. In yet another embodiment, the step of combining further comprising pumping from the continuous stirred-tank reactor to a second continuous stirred-tank reactor. In yet another embodiment, wherein the Michael adduct impurity is less than 1.5% as measured using the HPLC Analysis described herein. In yet another embodiment, wherein the Michael adduct impurity is less than 1% as measured using the HPLC Analysis described herein. In yet another embodiment, wherein the Michael adduct impurity is less than 0.75% as measured using the HPLC Analysis described herein. In yet another embodiment, wherein the Michael adduct impurity is less than 0.5% as measured using the HPLC Analysis described herein. In yet another embodiment, wherein the Michael adduct impurity is less than 0.4% as measured using the HPLC Analysis described herein. In yet another embodiment, wherein the Michael adduct impurity is less than 0.3% as measured using the HPLC Analysis described herein. In yet another embodiment, wherein the Michael adduct impurity is less than 0.25% as measured using the HPLC Analysis described herein. In yet another embodiment, further comprising a step of removing a Michael adduct impurity.

Certain stereochemical centers have been left unspecified and certain substituents have been eliminated in the following schemes for the sake of clarity and are not intended to limit the teaching of the schemes in any way. It will be understood that all stereoisomers are encompassed. Specific stereoisomers can be prepared by stereospecific synthesis using enantiomerically pure or enriched starting materials and/or reagents. Alternatively, enantiomers can be separated using methods known in the art, such as chiral chromatography or by converting the enantiomers to diastereomeric salts, separating the diastereomeric salts, converting the diastereomeric salt into a non-salt form and isolating the enantiomer. Individual isomers, enantiomers, and diastereomers may be separated or resolved by one of ordinary skill in the art at any convenient point in the synthesis of compounds of the disclosure, by methods such as selective crystallization techniques or chiral chromatography (See for example, J. Jacques, et al., “Enantiomers, Racemates, and Resolutions“, John Wiley and Sons, Inc., 1981, and E. L. Eliel and S. H. Wilen,” Stereochemistry of Organic Compounds”, Wiley-Interscience, 1994).

Additionally, the intermediates described in the following schemes contain a number of nitrogen or oxygen protecting groups. The variable protecting group may be the same or different in each occurrence depending on the particular reaction conditions and the particular transformations to be performed. The protection and deprotection conditions are well known to the skilled artisan and are described in the literature (See for example “Greene's Protective Groups in Organic Synthesis”, Fourth Edition, by Peter G. M. Wuts and Theodora W. Greene, John Wiley and Sons, Inc. 2007).

One of skill in the art will also appreciate that the compounds of Formulae I-IIa, described herein, or pharmaceutically acceptable salts thereof, can be deuterated (where a hydrogen can be replaced by a deuterium) and such molecules are within the scope of the compounds disclosed herein.

### Preparations and Examples

### Preparation 1

### Methyl 4-amino-3-chloro-2,5-difluoro-benzoate

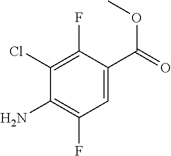

The title compound was prepared in the same manner as the method of the '877 and '633 references; Preparation 31. ES/MS m/z: 222 [M+H]⁺.

### Preparation 2

### Methyl 3-chloro-2,5-difluoro-4-iodo-benzoate

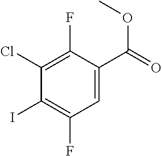

Cuprous iodide (22.7 g, 119 mmol), ACN (100 mL), and tert-butyl nitrite (17.7 mL, 149 mmol) were combined and stirred for 45 minutes at room temperature. A solution of methyl 4-amino-3-chloro-2,5-difluoro-benzoate (22.1 g, 100 mmol) in ACN (100 mL) was added. The reaction mixture was stirred at 40° C. for three hours, allowed to cool to room temperature, filtered through diatomaceous earth, and rinsed with DCM. The filtrate was concentrated in vacuo and purified by silica gel flash chromatography (10% EtOAc in hexanes) to give the title compound as a white solid (17.8 g, 54%). ¹H NMR (CDCl₃) S 7.60 (dd, J=5.6, 7.6 Hz, 1H), 3.98 (s, 3H).

### Preparation 3

### 3-Chloro-2,5-difluoro-4-iodo-benzoic acid

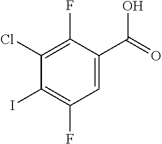 [SMILES: O=C(O)c1cc(F)c(I)c(Cl)c1F]

MeOH (50 mL) was added to a solution of methyl 3-chloro-2,5-difluoro-4-iodo-benzoate (16.8 g, 50.5 mmol) in THF (125 mL). The reaction mixture was cooled in an ice bath. 1 N aqueous NaOH (150 mL, 150 mmol) was added in three portions. The reaction mixture was allowed to stir at room temperature for 30 minutes and concentrated in vacuo to remove organic solvents. The aqueous remainder was cooled with ice chips and the pH adjusted to ˜2 with 5N aqueous HCl (25 mL). The aqueous layer was extracted with EtOAc (3×). The combined organic extracts were dried over sodium sulfate, filtered, and concentrated in vacuo to give the title compound (15.8 g, 98%). ¹H NMR S (DMSO-d₆) 13.91 (s, 1H), 7.68 (dd, J=5.8,7.8 Hz, 1H).

### Preparation 4

### tert-Butyl (3R)-4-(3-chloro-2,5-difluoro-4-iodo-benzoyl)-3-(hydroxymethyl)piperazine-1-carboxylate

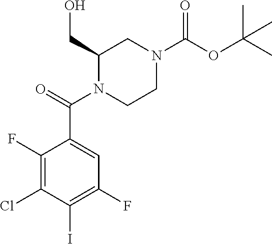 [SMILES: CC(C)(C)OC(=O)N1CCN(C(=O)c2cc(F)c(I)c(Cl)c2F)[C@@H](CO)C1]

HATU (3.31 g, 8.53 mmol) was added to a solution of 3-chloro-2,5-difluoro-4-iodo-benzoic acid (2.99 g, 9.39 mmol) and DIEA (6.7 mL, 38 mmol) in THF (100 mL). The reaction mixture was stirred at room temperature for one hour. A solution of tert-butyl (3R)-3-(hydroxymethyl)piperazine-1-carboxylate (1.92 g, 8.52 mmol) in THF (5 mL) was added dropwise. The reaction mixture was stirred at room temperature for 14 hours and 60° C. for three hours, then diluted with EtOAc and water. The organic layer was washed with water, 1 N aqueous NaOH, and brine. The aqueous layer was extracted with EtOAc, which was washed with brine. The combined organic extracts were dried over sodium sulfate, filtered, and concentrated in vacuo. The crude was purified by silica gel flash chromatography (10-50% EtOAc in hexanes) to give the title compound as a white solid (3.82 g, 82%). ES/MS m/z (³⁵Cl/³⁷Cl) 461/463 [M-tert-Butyl+H]⁺.

### Preparation 5

### tert-Butyl (13aS)-9-bromo-10-chloro-8-fluoro-6-oxo-1,3,4,12,13,13a-hexahydropyrazino[2,1-d][1,5]benzoxazocine-2-carboxylate

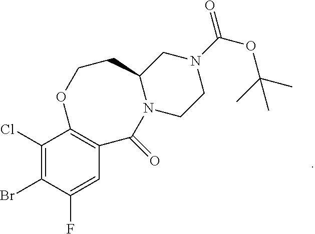 [SMILES: CC(C)(C)OC(=O)N1CCN2C(=O)c3cc(F)c(Br)c(Cl)c3OCC[C@H]2C1]

The title compound was prepared in the same manner as the method of the '877 and '633 references; Preparation 117. ES/MS m/z (⁷⁹Br/⁸¹Br) 407/409 [M-tert-Butyl+H]⁺.

### Preparation 20

### tert-Butyl (3S)-4-(4-bromo-2,5-difluoro-benzoyl)-3-(2-hydroxyethyl)piperazine-1-carboxylate

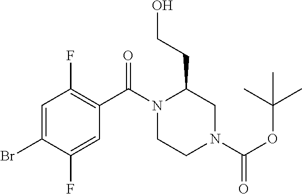 [SMILES: CC(C)(C)OC(=O)N1CCN(C(=O)c2cc(F)c(Br)cc2F)[C@@H](CCO)C1]

CDMT (802.8 g, 4.57 mol) was added to a reactor along with acetonitrile (20.0 L) and the mixture was cooled to 0° C. NMM (708.2 g, 7.00 mol) was added and the mixture was stirred for 30 min at 0° C. 4-bromo-2,5-difluorobenzoic acid (794.8 g, 3.35 mol),

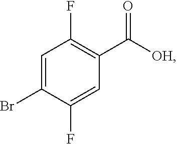 [SMILES: O=C(O)c1cc(F)c(Br)cc1F]

was added and the mixture was stirred for 1 h at 0° C. In a separate vessel, K₂CO₃ (842 g, 6.09 mol), water (5.0 L), and tert-butyl (3S)-3-(2-hydroxyethyl)piperazine-1-carboxylate:phosphoric acid (1:1) (1000 g, 3.05 mol) were combined. The aqueous piperazine solution was added to the acetonitrile coupling solution over 5 min while maintaining an internal temperature of 0° C. The mixture was stirred for 2.5 h at 0° C. The bottom layer was drained from the reactor. The reaction mixture was stirred for an additional 64 h at 0° C., then concentrated to 2 L/kg. 2-MeTHF (10.0 L) and n-heptane (3.0 L) were added. 1 M aq. NaHCO₃ (10.0 L) was added and the mixture was stirred for 15 min. The layers were allowed to separate and the bottom layer was drained. 1 M aq. NaHCO₃ (10.0 L) was added and the mixture was stirred for 15 min. The layers were allowed to separate and the bottom layer was drained. Aq. HCl (0.05 M, 5.0 L) was added and the mixture was stirred for 15 min. The layers were allowed to separate and the bottom layer was drained. Water (5.0 L) was added and the mixture was stirred for 15 min. The layers were allowed to separate and the bottom layer was drained. The upper layer was concentrated to 2 L/kg and 2-MeTHF (1.0 L) was added. The mixture was concentrated to 2 L/kg and 2-MeTHF (1.0 L) was added. The mixture was concentrated to 2 L/kg and DMF (2.7 L) was added to give the title compound as a DMF solution (4.03 kg, 31.2% assay, 2.80 mol, 92% yield), which was used without further manipulation. An analytical sample was prepared by chromatography and exhibited the following spectroscopic characteristics: APCI-MS found 471 and 473 (M+Na⁺); ¹H NMR (500 MHz, d₆-DMSO+TFA, multiple rotamers present) S 7.84 (m, 1H), 7.53 (br s, 1H), 4.71 (br s, 0.5H), 4.33-4.27 (m, 0.5H), 4.12-3.72 (m, 2H), 3.65 (br s, 0.5H), 3.47 (br, 1H), 3.38-3.11 (m, 2H), 3.11-2.56 (m, 2.5H), 1.68 (m, 2H), 1.39 (s 9H).

### Preparation 21

### tert-Butyl (13aS)-9-bromo-8-fluoro-6-oxo-1,3,4,12,13,13a-hexahydropyrazino[2,1-d][1,5]benzoxazocine-2-carboxylate

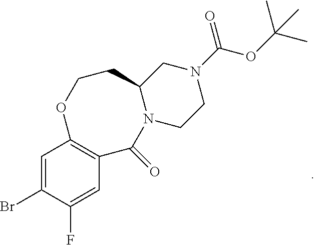 [SMILES: CC(C)(C)OC(=O)N1CCN2C(=O)c3cc(F)c(Br)cc3OCC[C@H]2C1]

Sodium tert-pentoxide (462 g, 4.20 mol) was added to a reactor along with DMF (9.1 L). The mixture was stirred to form a solution and cooled to 0° C. The DMF solution of tert-butyl (3S)-4-(4-bromo-2,5-difluoro-benzoyl)-3-(2-hydroxyethyl)piperazine-1-carboxylate (4.03 kg, 31.2% assay, 2.80 mol) was added over 8 h at 0° C. The total mass of the product solution at this point is 12.95 kg. A portion of the solution (1.30 kg) was placed in a separate reactor. Water (2.6 kg) was added to a clean reactor. The 1.30 kg of product solution was added to the water over 3 h at 25° C. The mixture was stirred for 64 h at 25° C. The resulting solids were isolated by filtration and washed with a mixture of DMF (0.3 L) and water (0.3 L). The crude solids were added to a reactor along with EtOH (628 mL). The mixture was heated to 55° C. and stirred for 30 min. Water (314 mL) was added over 2 h and the mixture was held at 55° C. for an additional 30 min. The slurry was cooled to 20° C. over 2 h, then held at 20° C. for 2 h. The resulting solids were isolated by filtration and washed with a mixture of water (60 mL) and EtOH (60 mL). The product was dried under vacuum at 50° C. for 16 h to give the title compound (98.2 g, 73% yield). APCI-MS found 451 and 453 (M+Na⁺¹); ¹H NMR (400 MHz, d₆-DMSO-) S 7.32 (d, J=6.0 Hz, 1H), 7.28 (d, J=9.2 Hz, 1H), 4.24-4.18 (m, 2H), 4.05-3.91 (m, 2H), 3.87-3.76 (m, 2H), 3.06 (br, 1H), 2.95-2.80 (brm, 2H), 2.06 (m, 1H), 1.76 (m, 1H), 1.04 (s, 9H).

### Alternative Synthesis of Preparation 5

### tert-Butyl (13aS)-9-bromo-10-chloro-8-fluoro-6-oxo-1,3,4,12,13,13a-hexahydropyrazino[2,1-d][1,5]benzoxazocine-2-carboxylate

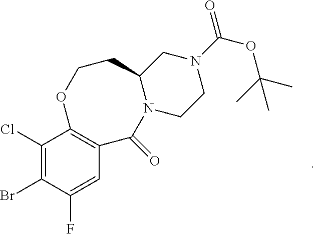 [SMILES: CC(C)(C)OC(=O)N1CCN2C(=O)c3cc(F)c(Br)c(Cl)c3OCC[C@H]2C1]

tert-Butyl (13aS)-9-bromo-8-fluoro-6-oxo-1,3,4,12,13,13a-hexahydropyrazino[2,1-d][1,5]benzoxazocine-2-carboxylate (2.00 g, 4.66 mmol) was added to a reactor along with acetonitrile (20 mL) and the mixture was cooled to 5° C. Trichloroisocyanuric acid (1.08 g, 4.66 mmol) was added and the mixture was stirred at 5° C. for 5 min. A solution of phosphoric acid (0.96 mL, 14.0 mmol; stock solution was 1.92 mL of phosphoric acid in 4.0 mL of acetonitrile) was added over 2 min and the mixture was stirred at 5° C. for 23 h. Aq. K₂CO₃ (40%, 20 mL) was added and the mixture was stirred at 5° C. for 30 min, warmed to 20° C., and stirred at 20° C. for 2 h. Ethyl acetate (20 mL) and water (20 mL) were added and the mixture was stirred until solids dissolved. Solids reappeared after sitting for 1 h. Aq. NaHSO₃ (5%, 10 mL) was added and the mixture was stirred overnight. Aq. K₂CO₃ (40%, 4 mL) and di-tert-butyl dicarbonate (0.20 g, 0.93 mmol) were added and the mixture was stirred overnight. The mixture was concentrated to 2-3 volumes and isopropanol (4 volumes) was added. The mixture was concentrated to 2-3 volumes and isopropanol (4 volumes) was added. The mixture was concentrated to 2-3 volumes and isopropanol (2 volumes) was added. The mixture was stirred at 57° C. Water (4 volumes) was added over 1 h and the mixture was stirred at 57° C. for 2 h. The mixture was cooled and stirred overnight at 20° C., filtered, and washed with 1:2 isopropanol:water (3 volumes). The product was dried under vacuum at 50° C. for 3 h to give the title compound (1.85 g, 86% yield).

### Preparation 6

### tert-Butyl (4aR)-7-chloro-9-fluoro-8-iodo-11-oxo-2,4,4a,5-tetrahydro-1H-pyrazino[2,1-c][1,4]benzoxazepine-3-carboxylate

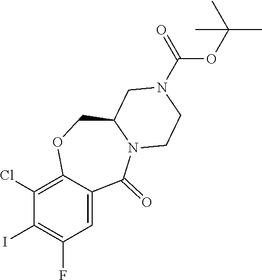 [SMILES: CC(C)(C)OC(=O)N1CCN2C(=O)c3cc(F)c(I)c(Cl)c3OC[C@H]2C1]

tert-Butyl (3R)-4-(3-chloro-2,5-difluoro-4-iodo-benzoyl)-3-(hydroxymethyl)piperazine-1-carboxylate (4.59 g, 8.88 mmol) in DMF (150 mL) was cooled to 0° C. Sodium hydride (0.89 g, 22 mmol, 60 mass % in paraffin oil) was added. After four hours at 0° C., the reaction mixture was quenched with saturated aqueous sodium bicarbonate and diluted with water and diethyl ether. The aqueous layer was extracted with diethyl ether (4×). The combined organic extracts were washed with water (3×) and brine, dried over sodium sulfate, filtered, and concentrated in vacuo. The crude was purified by silica gel flash chromatography (15-40% EtOAc in hexanes) to give the title compound as a white solid (3.82 g, 82%). ES/MS m/z (³⁵Cl/³⁷Cl) 441/443 [M-tert-Butyl+H]⁺.

### Preparation 7

### tert-Butyl N-(4-bromo-3-cyano-7-fluoro-benzothiophen-2-yl)carbamate

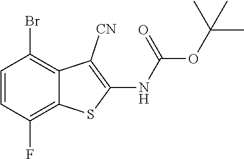 [SMILES: CC(C)(C)OC(=O)Nc1sc2c(F)ccc(Br)c2c1C#N]

The title compound was prepared in the same manner as the method of the '877 and '633 references; Preparation 14. ES/MS m/z (⁷⁹Br/⁸¹Br) 315/317 [M-tert-Butyl+H]⁺.

### Preparation 8

### tert-Butyl N-[3-cyano-7-fluoro-4-(4,4,5,5-tetramethyl-1,3,2-dioxaborolan-2-yl)benzothiophen-2-yl]carbamate

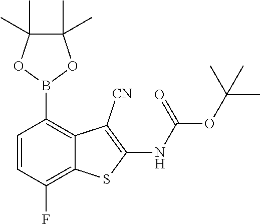 [SMILES: CC(C)(C)OC(=O)Nc1sc2c(F)ccc(B3OC(C)(C)C(C)(C)O3)c2c1C#N]

A mixture of tert-butyl N-(4-bromo-3-cyano-7-fluoro-benzothiophen-2-yl)carbamate (3.10 g, 8.35 mmol), bis(pinacolato)diboron (21.0 g, 82.7 mmol), and potassium acetate (2.50 g, 25.5 mmol) in 1,4-dioxane (50 mL) was flushed with nitrogen (direct sparge) for 10 minutes. 1,1′-Bis(diphenylphosphino)ferrocene-palladium(II)dichloride dichloromethane complex (1.0 g, 1.2 mmol) was added. The reaction flask was sealed and heated at 80-85° C. for 3.5 hours. The reaction mixture was filtered through diatomaceous earth. The filtrate was diluted with water and extracted with EtOAc (2×). The combined organic extracts were washed with brine, dried over sodium sulfate, filtered, and concentrated in vacuo. The crude was purified by silica gel flash chromatography (10-50% (20% acetone in DCM) in hexanes) to give the title compound (4.45 g, 87%). ES/MS m/z 363 [M-tert-Butyl+H]⁺.

### Preparation 9

### tert-Butyl N-[3-cyano-4-(5,5-dimethyl-1,3,2-dioxaborinan-2-yl)-7-fluoro-benzothiophen-2-yl]carbamate

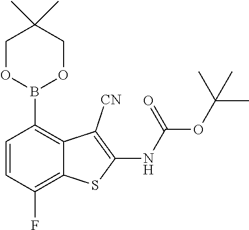 [SMILES: CC1(C)COB(c2ccc(F)c3sc(NC(=O)OC(C)(C)C)c(C#N)c23)OC1]

The title compound was prepared in the same manner as the method of the '877 and '633 references; Preparation 15. ¹H NMR (DMSO-d₆) δ 11.6 (s, 1H), 7.61 (m, 1H), 7.20 (m, 1H), 3.78 (s, 4H), 1.54 (s, 9H), 1.03 (s, 6H).

### Preparations 10 and 11

### tert-Butyl (13aS)-9-[2-(tert-butoxycarbonylamino)-3-cyano-benzothiophen-4-yl]-10-chloro-8-fluoro-6-oxo-1,3,4,12,13,13a-hexahydropyrazino[2,1-d][1,5]benzoxazocine-2-carboxylate, P and M atropisomers

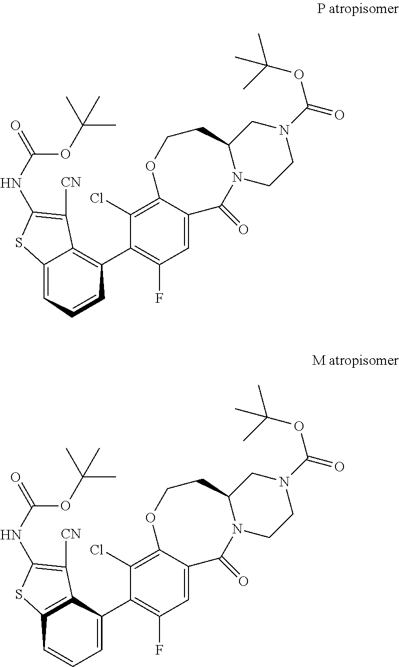

The title compounds were prepared in the same manner as the method of the '877 and '633 references; Preparation 167. The mixture of atropisomers were separated by silica gel flash chromatography (0-30% acetone in hexanes). Preparation 10 (P atropisomer) was the first compound to elute off the column. Preparation 11 (M atropisomer) was the second compound to elute off the column. For both, ES/MS m/z (³⁵Cl/³⁷Cl) 657/659 [M+H]⁺.

### Preparations 12 and 13

### tert-Butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, P and M atropisomers

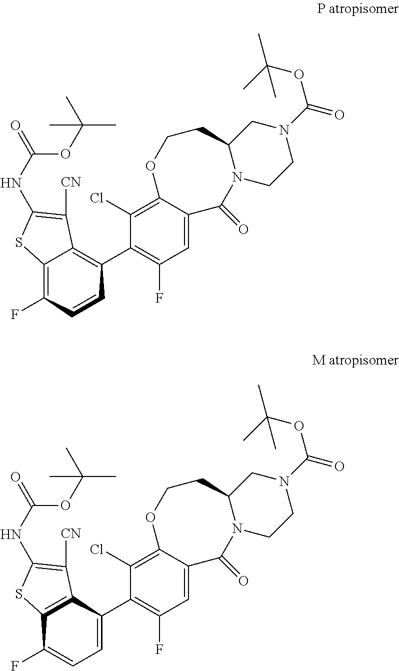

A mixture of tert-butyl (13aS)-9-bromo-10-chloro-8-fluoro-6-oxo-1,3,4,12,13,13a-hexahydropyrazino[2,1-d][1,5]benzoxazocine-2-carboxylate (10.0 g, 21.6 mmol), tert-butyl N-[3-cyano-4-(5,5-dimethyl-1,3,2-dioxaborinan-2-yl)-7-fluoro-benzothiophen-2-yl]carbamate (12.3 g, 30.4 mmol), potassium carbonate (8.94 g, 64.7 mmol), and dichloro[(S)-(−)-2,2′-bis(diphenylphosphino)-1,1′-binaphthyl]palladium(II) (also known as “(S)-(−)-2,2′-bis(diphenylphosphino)-1,1′-binaphthyl palladium dichloride”) (3.84 g, 4.42 mmol) was added to 1,4-dioxane (215 mL) that had been flushed with nitrogen (direct sparge) for 30 minutes. The reaction flask was sealed and heated at 105° C. for 14 hours. Additional tert-butyl N-[3-cyano-4-(5,5-dimethyl-1,3,2-dioxaborinan-2-yl)-7-fluoro-benzothiophen-2-yl]carbamate (3.54 g, 8.75 mmol) and dichloro[(S)-(−)-2,2′-bis(diphenylphosphino)-1,1′-binaphthyl]palladium(II) (0.97 g, 1.12 mmol) were added. The reaction flask was sealed and heated at 105° C. for 14 hours.

The reaction mixture was filtered through diatomaceous earth, and rinsed with EtOAc. The filtrate was concentrated in vacuo and diluted with EtOAc, water, and brine. The aqueous layer was extracted with EtOAc. The combined organic extracts were dried over magnesium sulfate, filtered, and concentrated in vacuo. The crude was purified by silica gel flash chromatography (10-40% acetone in hexanes). Preparation 12 (P atropisomer) eluted first, followed by Preparation 13 (M atropisomer). Impure fractions of P atropisomer were further purified by silica gel flash chromatography. This gave the two title compounds (P atropisomer, 0.35 g, 2%; M atropisomer, 5.6 g, 38%). For both, ES/MS m/z 619 [M-tert-Butyl+H]⁺.

### Catalyst Screen of Preparations 12 and 13

### tert-Butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, P and M atropisomers

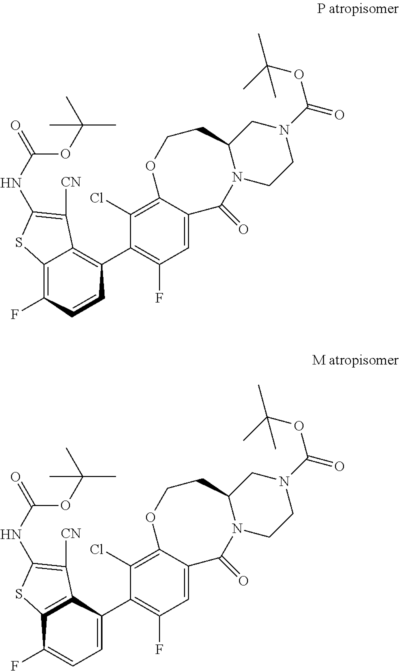

In a glove box, a mixture of tert-butyl (13aS)-9-bromo-10-chloro-8-fluoro-6-oxo-1,3,4,12,13,13a-hexahydropyrazino[2,1-d][1,5]benzoxazocine-2-carboxylate (0.025 g, 0.054 mmol), tert-butyl N-[3-cyano-4-(5,5-dimethyl-1,3,2-dioxaborinan-2-yl)-7-fluoro-benzothiophen-2-yl]carbamate (0.030 g, 0.074 mmol), potassium carbonate (0.022 g, 0.16 mmol), and catalyst (0.0081 mmol) was added to 1,4-dioxane (1 mL) in a vial. The vial was sealed and heated at 105° C. for 19 hours, then cooled to room temperature. Reaction monitoring by LCMS showed the following reaction progression and ratio of atropisomers:

TABLE 1

| | Reaction | Ratio of |
|---|---|---|
| | Progression (% | Prep 12:Prep 13 |
| | loss of limiting | P:M |
| Catalyst | reactant) | atropisomers |
| Dichloro[(S)-(−)-2,2′-bis(diphenylphosphino)- | 100 | 1:4 |
| 1,1′-binaphthyl]palladium(II) | | |
| Dichloro[(R)-(+)-2,2′-bis(diphenylphosphino)- | 100 | 1:2 |
| 1,1′-binaphthyl]palladium(II) | | |
| Dichloro 1,1′-bis(diphenylphosphino)ferrocene | 100 | 1:2 |
| palladium (II) dichloromethane | | |
| Dichloro(1,2- | 71 | 1:2 |
| bis(diphenylphosphino)ethane)palladium(II) | | |
| Dichloro(1,3- | 56 | 1:2 |
| bis(diphenylphosphino)propane)palladium(II) | | |
| Dichloro[1,4- | 85 | 1:2 |
| bis(diphenylphosphino)butane]palladium(II) | | |
| Dichloro{bis[2- | 100 | 2:3 |
| (diphenylphosphino)phenyl]ether}palladium(II) | | |
| Dichloro[9,9-dimethyl-4,5- | 4 | |
| bis(diphenylphosphino)xanthene]palladium(II) | | |
| Dibromo[1,1′- | 100 | 1:2 |
| bis(diphenylphosphino)ferrocene]palladium(II) | | |
| [(1,2,3-η)-2-Buten-1-yl]chloro[dicyclohexyl(2′,6′- | 28 | 1:4 |
| dimethoxy[1,1′-biphenyl]-2-yl)phosphine- | | |
| κP]palladium | | |
| [(1,2,3-η)-2- | 100 | 2:3 |
| Butenyl]chloro(tricyclohexylphosphine)palladium | | |
| trans- | 21 | |
| Dichlorobis(tricyclohexylphosphine)palladium(II) | | |

### Alternative Synthesis of Preparation 13

### tert-Butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, M atropisomer

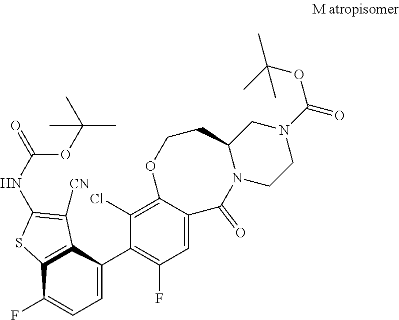

To a small flask, in a glove box, was added palladium (II) acetate (0.119 g, 0.528 mmol), (S)-(−)-2,2′-bis(diphenylphosphino)-1,1′-binaphthalene (0.364 g, 0.580 mmol) and toluene (25 mL), which would make diacetate[(S)-(−)-2,2′-bis(diphenylphosphino)-1,1′-binaphthyl]palladium(II). The mixture was capped and stirred for 1 hour. tert-Butyl (13aS)-9-bromo-10-chloro-8-fluoro-6-oxo-1,3,4,12,13,13a-hexahydropyrazino[2,1-d][1,5]benzoxazocine-2-carboxylate (0.500 g, 1.06 mmol) and a solution of potassium carbonate in water (2 mL, made from 0.241 g potassium carbonate in 2 mL water) were added. The mixture was heated at 60° C. for 6 hours. The organic and aqueous layers were separated. The organic layer was dried over magnesium sulfate and filtered. The solids were rinsed with toluene (25 mL), which was added to the original filtrate.

To a second flask was added tert-butyl (13aS)-9-bromo-10-chloro-8-fluoro-6-oxo-1,3,4,12,13,13a-hexahydropyrazino[2,1-d][1,5]benzoxazocine-2-carboxylate (9.51 g, 20.16 mmol) and cesium carbonate (31.1 g, 95.0 mmol). The flask was cycled through vacuum and nitrogen four times.

To a third flask was added tert-butyl N-[3-cyano-4-(5,5-dimethyl-1,3,2-dioxaborinan-2-yl)-7-fluoro-benzothiophen-2-yl]carbamate (10.9 g, 25.3 mmol), 1,1,1-tris(hydroxymethyl)ethane (1.69 g), and THF (50 mL). The mixture was stirred for 25 min.

To the second flask was added the ligand-catalyst filtrate and THF (50 mL). The mixture was heated at 60° C. under nitrogen. The contents of the third flask were added dropwise (6 mL/hour). After 10 total hours of heating, HPLC analysis showed complete conversion of bromide starting material to product in a 4.36:1 M:P atropisomer ratio.

After 12 total hours of heating, the mixture was allowed to cool to room temperature. Water (100 mL) was added and the organic and aqueous layers were separated. 20% brine (50 mL) was added and the organic and aqueous layers were separated. A portion (˜20%) of the organic layer (26.2 g) was distilled to −10 mL, diluted with isopropyl acetate (20 mL), distilled to −10 mL, diluted with isopropyl acetate (20 mL), and distilled to ˜10 mL. The resulting suspension was filtered and dried under vacuum to give the title compound (1.85 g). HPLC analysis showed 98.23% M atropisomer and 0.76% P atropisomer.

### Preparations 14 and 15

### tert-Butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate, P and M atropisomers

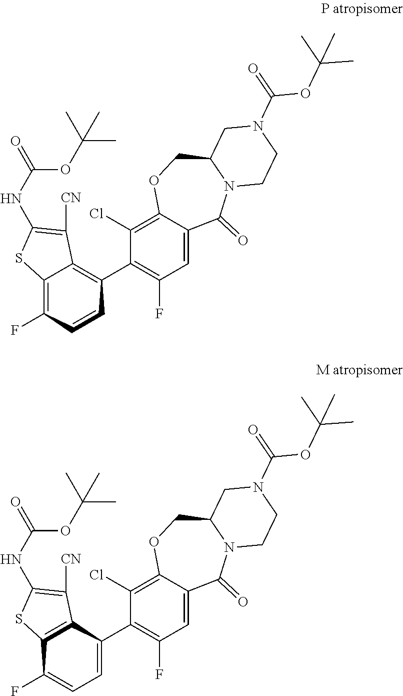

A solution of tert-butyl (4aR)-7-chloro-9-fluoro-8-iodo-11-oxo-2,4,4a,5-tetrahydro-1H-pyrazino[2,1-c][1,4]benzoxazepine-3-carboxylate (0.50 g, 1.01 mmol), tert-butyl N-[3-cyano-7-fluoro-4-(4,4,5,5-tetramethyl-1,3,2-dioxaborolan-2-yl)benzothiophen-2-yl]carbamate (0.63 g, 1.51 mmol), and potassium phosphate (0.50 g, 2.36 mmol) in water (2 mL) and 1,4-dioxane (10 mL) was flushed with nitrogen (direct sparge) for 10 minutes. 1,1′-Bis(di-tert-butylphosphino)ferrocene palladium dichloride (0.20 g, 0.30 mmol) was added. The reaction flask was sealed and heated at 70° C. for three hours.

A solution of tert-butyl (4aR)-7-chloro-9-fluoro-8-iodo-11-oxo-2,4,4a,5-tetrahydro-1H-pyrazino[2,1-c][1,4]benzoxazepine-3-carboxylate (0.20 g, 0.40 mmol), tert-butyl N-[3-cyano-7-fluoro-4-(4,4,5,5-tetramethyl-1,3,2-dioxaborolan-2-yl)benzothiophen-2-yl]carbamate (0.24 g, 0.57 mmol), and potassium phosphate (0.20 g, 0.96 mmol) in water (2.5 mL) and 1,4-dioxane (8 mL) was flushed with nitrogen (direct sparge) for 10 minutes. 1,1′-Bis(di-tert-butylphosphino)ferrocene palladium dichloride (0.080 g, 0.12 mmol) was added. The reaction flask was sealed and heated at 70° C. for five hours.

The two reaction mixtures were combined, filtered through diatomaceous earth, and rinsed with EtOAc. The filtrate was diluted with MTBE and saturated aqueous sodium bicarbonate. The organic extract was washed with brine, dried over magnesium sulfate, filtered, and concentrated in vacuo. The crude was purified by silica gel flash chromatography (0-30% acetone in hexanes). Preparation 14 (P atropisomer) eluted first, followed by Preparation 15 (M atropisomer). Impure fractions of P atropisomer were further purified by silica gel flash chromatography (0-100% EtOAc in hexanes). This gave the two title compounds (P atropisomer, 0.17 g, 17%; M atropisomer, 0.21 g, 21%). For both, ES/MS m/z (³⁵Cl/³⁷Cl) 605/607 [M-tert-Butyl+H]⁺.

### Preparation 16

### 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile, P atropisomer

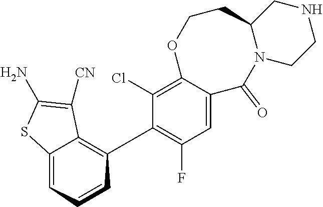

TFA (4 mL) was added to a solution of tert-butyl (13aS)-9-[2-(tert-butoxycarbonylamino)-3-cyano-benzothiophen-4-yl]-10-chloro-8-fluoro-6-oxo-1,3,4,12,13,13a-hexahydropyrazino[2,1-d][1,5]benzoxazocine-2-carboxylate, P atropisomer (0.98 g, 1.5 mmol) in DCM (5 mL). The reaction mixture was stirred at room temperature for three hours, concentrated in vacuo, and purified by silica gel flash chromatography (4-10% 7N ammoniated MeOH in DCM) to give the title compound (0.57 g, 84%). ES/MS m/z (³⁵Cl/³⁷Cl) 457/459 [M+H]⁺.

### Preparation 17

### 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile, M atropisomer

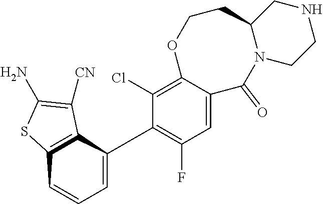

The title compound was prepared in the same manner as the method of the '877 and '633 references; Preparation 187. ES/MS m/z (³⁵Cl/³⁷Cl) 457/459 [M+H]⁺.

### Preparation 18

### 4-[(4aR)-7-Chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, P atropisomer

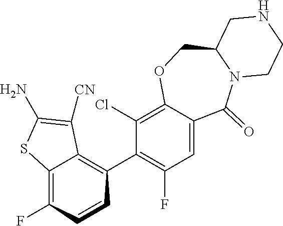

TFA (2 mL) was added to a solution of tert-butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate, P atropisomer (0.018 g, 0.027 mmol) in DCM (2 mL). The reaction mixture was stirred at room temperature for 30 minutes, concentrated in vacuo, and purified by silica gel flash chromatography (0-10% MeOH in DCM, then 0-10% 7N ammoniated MeOH in DCM) to give the title compound (0.013 g, 100%). ES/MS m/z (³⁵Cl/³⁷Cl) 461/463 [M+H]⁺.

### Preparation 19

### 4-[(4aR)-7-Chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, M atropisomer

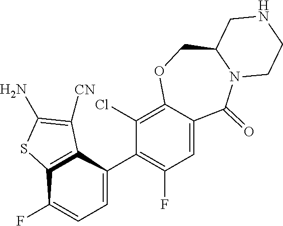

TFA (2.5 mL) was added to an ice-cooled solution of tert-butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate, M atropisomer (0.20 g, 0.31 mmol) in DCM (2.5 mL). The reaction mixture was stirred at room temperature for two hours, concentrated in vacuo, and purified by silica gel flash chromatography (0-10% 7N ammoniated MeOH in DCM) to give the title compound (0.13 g, 89%). ES/MS m/z (³⁵Cl/³⁷Cl) 461/463 [M+H]⁺.

### Preparation 22

### 4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer

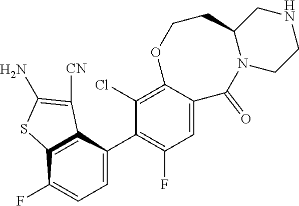

tert-Butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, M atropisomer (100.0 g, 148.1 mmol) was added to a reactor with EtOH (300 mL). A solution of HCl in EtOH (30%, 216 g, 1.78 mol) was added over 30 min with stirring. The mixture was heated to 40° C. for 20 h, then cooled to 0° C. with stirring. A mixture of KOH (118.8 g, 1.80 mol) in water (1.0 L) was gradually added with stirring. The EtOH was removed by distillation. The residue was extracted with 2-MeTHF (1.0 L) and again with 2-MeTHF (500 mL). The organic extracts were combined, washed with saturated aqueous NaCl (3×0.5 L), and concentrated to −200 mL. Acetonitrile (1.0 L) was added in a dropwise manner. The mixture was stirred and cooled to 0° C. for 1 h. The solids were collected by filtration, washed with acetonitrile (200 mL), and dried under vacuum to afford the title compound as a white solid (66.4 g, 85% yield). APCI-MS 475 (M+H⁺).

### Examples 1 and 2

### (13aS)-9-(2-Amino-7-fluoro-1,3-benzothiazol-4-yl)-8,10-dichloro-2-prop-2-enoyl-1,3,4,12,13,13a-hexahydropyrazino[2,1-d][1,5]benzoxazocin-6-one, P and M atropisomers

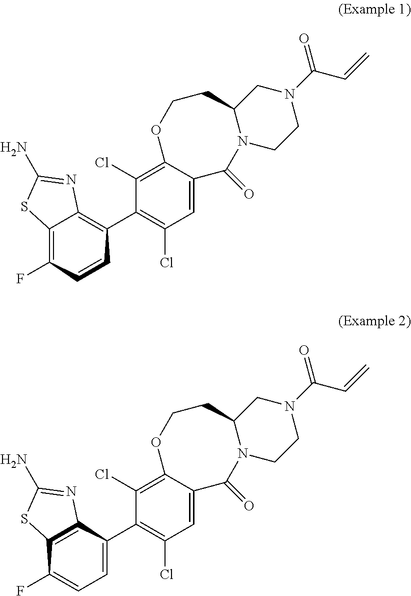

The title compounds were prepared in the same manner as the method of the '877 and '633 references; Example 1. The mixture of atropisomers was separated using Chiralpak® IC, 4.6×150 mm, 40% EtOH/CO₂, 5 mL/min, 225 nm. Example 1 (P atropisomer) is the first compound off the column. Example 2 (M atropisomer) is the second compound off the column. For both, ES/MS m/z (³⁵Cl/³⁷Cl) 521/523 [M+H]⁺.

### Example 3

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-benzothiophene-3-carbonitrile, P atropisomer

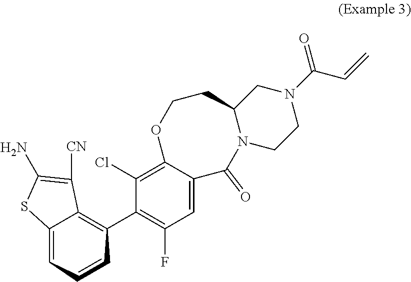

### Example 3

A solution of 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile, P atropisomer (0.115 g, 0.252 mmol) and DIEA (0.20 mL, 1.2 mmol) in DCM (2 mL) was cooled to −78° C. Acryloyl chloride (0.02 mL, 0.2 mmol) was added. After five minutes, diluted with a small amount of 2-propanol and concentrated in vacuo. The crude was purified by silica gel flash chromatography (20-100% acetone in hexanes) to give the title compound (0.091 g, 71%). ES/MS m/z (³⁵Cl/³⁷Cl) 511/513 [M+H]⁺.

### Example 4

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-benzothiophene-3-carbonitrile, M atropisomer

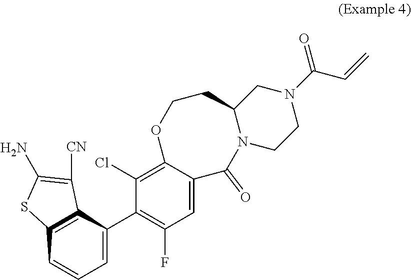

### Example 4

The title compound was prepared in the same manner as the method of the '877 and '633 references; Example 34. ES/MS m/z (³⁵Cl/³⁷Cl) 511/513 [M+H]⁺.

### Example 5

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, P atropisomer

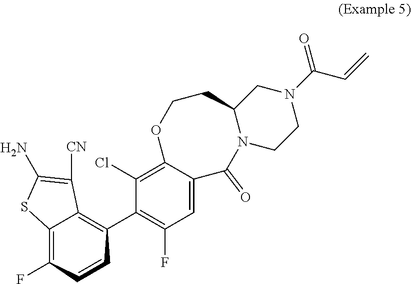

TFA (1 mL) was added to a solution of tert-butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, P atropisomer (0.025 g, 0.037 mmol) in DCM (1 mL). The reaction mixture was stirred at room temperature for 30 minutes, then concentrated in vacuo to give crude deprotected material.

The residue was dissolved in DCM (1 mL) and DIEA (0.013 mL, 0.074 mmol) was added. The solution was cooled to −78° C. Acryloyl chloride (0.0030 mL) in DCM (1 mL) was added. After 30 minutes, concentrated in vacuo. The crude was purified by silica gel flash chromatography (20-80% acetone in hexanes) to give the title compound (0.012 g, 61%). ES/MS m/z (³⁵Cl/³⁷Cl) 529/531 [M+H]⁺.

### Example 6

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, M atropisomer

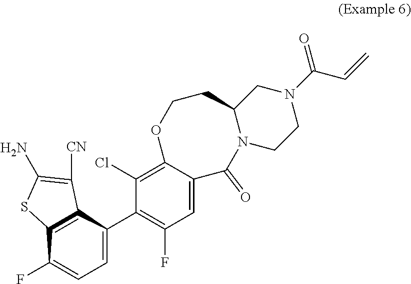

HCl gas was bubbled for five minutes into an ice-cooled solution of tert-butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate, M atropisomer (0.786 g, 1.17 mmol) in DCM (12 mL) and 2-propanol (12 mL). The reaction mixture was stirred at room temperature for five hours, then cooled in an ice bath. HCl gas was bubbled into the reaction mixture for five minutes. The reaction mixture was stirred at room temperature for 14 hours, then concentrated in vacuo. The residue was twice diluted with n-heptane and concentrated in vacuo. MTBE (50 mL) was added. The mixture was stirred at room temperature for ten minutes, then filtered to give the deprotected material as a dihydrochloride salt.

The dihydrochloride salt was dissolved in water (12 mL). 2-Methyltetrahydrofuran (12 mL) was added. A solution of potassium carbonate (0.81 g, 5.82 mmol) in water (12 mL) was added. The mixture was vigorously stirred while being cooled in an ice bath. Acryloyl chloride (0.10 mL, 1.28 mmol) in 2-methyltetrahydrofuran (5 mL) was added dropwise. After five minutes, diluted with brine and 2-methyltetrahydrofuran. The aqueous layer was extracted with 2-methyltetrahydrofuran (2×). The combined organic extracts were washed with water, dried over magnesium sulfate, filtered, and concentrated in vacuo. The crude was purified by silica gel flash chromatography (20-30% B in A; A:3:1 EtOAc:hexanes; B: 4:1 EtOAc:MeOH) to give the title compound (0.409 g, 66%). ES/MS m/z (³⁵Cl/³⁷Cl) 529/531 [M+H]⁺.

### Alternative Synthesis of Example 6: Flow Chemistry

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, M atropisomer

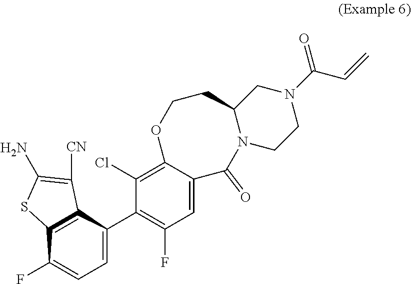

### Example 6

To a continuous stirred-tank reactor (105 mL, 15° C.) was added material from feed A [monopotassium phosphate (25.02 g), dipotassium phosphate (2.61 g), and water (to 600 mL volume); pH˜5.8; flow rate 4.73 g/min], feed B [acryloyl chloride (13.15 g, 1.15 eq) and 2-methyltetrahydrofuran (6 volumes); flow rate 2.48 g/min], and feed C [4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer (60 g, 0.126 mol), 2-methyltetrahydrofuran (6 volumes), and water (0.2 volumes); flow rate 2.97 g/min]. The mixture in the reactor was pumped to a second continuous stirred-tank reactor (105 mL, 15° C.) at a flow rate of 10.5 g/min, and then collected in a separate receiving bottle. HPLC analysis conditions (“HPLC analysis”) comprise an Inertsil ODS-3V column (4.6×250 mm; 5 μm) eluted with 20% to 95% ACN in 0.1% aqueous H₃PO₄ using an Agilent 1260 chromatograph with UV detector. HPLC analysis showed 0.33% impurity, 4-[(13aS)-2-[3-[(13aS)-9-(2-amino-3-cyano-7-fluoro-benzothiophen-4-yl)-10-chloro-8-fluoro-6-oxo-1,3,4,12,13,13a-hexahydropyrazino[2,1-d][1,5]benzoxazocin-2-yl]-3-oxo-propyl]-10-chloro-8-fluoro-6-oxo-1,3,4,12,13,13a-hexahydropyrazino[2,1-d][1,5]benzoxazocin-9-yl]-2-amino-7-fluoro-benzothiophene-3-carbonitrile, M,M atropisomer,

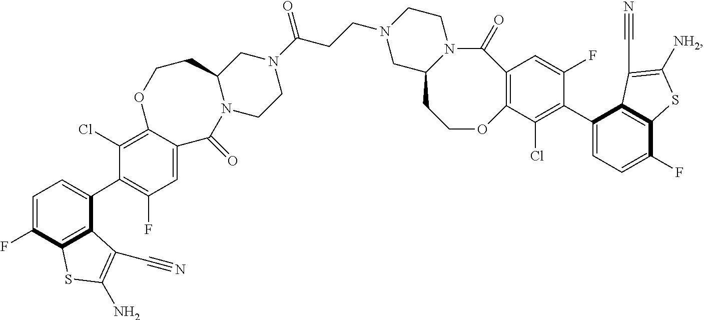

“MA impurity” as shown in Table 2. This MA impurity is a Michael adduct wherein unreacted [4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer reacts with the compound of formula I and thereby forms a dimer. It is possible for trace amounts of the corresponding M,P atropisomer dimer may be present. The lower percentage of this impurity in a flow reaction relative to the higher percentage of this impurity in a comparison batch reaction was surprising and unexpected. The final mixture layers were separated. The aqueous phase was extracted with 2-methyltetrahydrofuran (5 volumes). The combined organic phases were washed with aqueous sodium bicarbonate (5 wt %, 10 volumes) twice and aqueous sodium chloride (10%, 10 volumes) once. The organic phase was concentrated to 10 volumes, diluted with isopropanol (10 volumes), concentrated to 10 volumes, diluted with isopropanol (10 volumes), stirred at 15° C. for 2 hours, filtered, washed, and dried to give crude title compound (˜60 g).

Crude title compound (˜50 g) was dissolved in 4:1 THF:MeOH (11 volumes) at 35° C., diluted with methylcyclohexane (0.58 volumes), seeded (2.5 wt %), and stirred for 30 min. Methylcyclohexane (12.3 volumes) was added steadily over 10-12 hours. The mixture was stirred overnight at 35° C., filtered, washed with methylcyclohexane (2 volumes), and dried to give the title compound (46.4 g, 84% adjusted yield).

### Alternative Synthesis of Example 6: Batch Chemistry

### 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile, M atropisomer

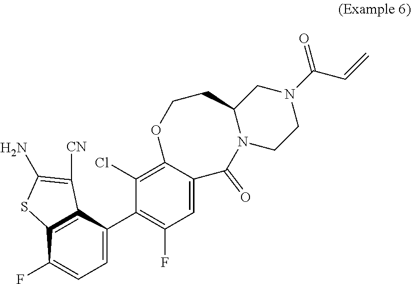

A batch reaction (one-sixth scale) was carried out, in which a solution of acryloyl chloride (2.19 g, 1.15 eq) in 2-methyltetrahydrofuran (6 volumes) was added in a dropwise manner over 30 min to a solution of 4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile, M atropisomer (10 g, 21 mmol) in 2-methyltetrahydrofuran (6 volumes) and pH 5.8 buffer [monopotassium phosphate (4.17 g), dipotassium phosphate (0.435 g), and water (to 100 mL volume)]. HPLC analysis after 25 min showed 1.63% MA impurity as shown in Table 2.

TABLE 2

| Alternative Synthesis of Example 6 | % MA impurity |
|---|---|
| Flow Chemistry | 0.33 |
| Batch Chemistry | 1.63 |

### Example 7

### 4-[(4aR)-7-Chloro-9-fluoro-11-oxo-3-prop-2-enoyl-2,4,4a,5-tetrahydro-1H-pyrazino[2,1-c][1,4]benzoxazepin-8-yl]-2-amino-7-fluoro-benzothiophene-3-carbonitrile, P atropisomer

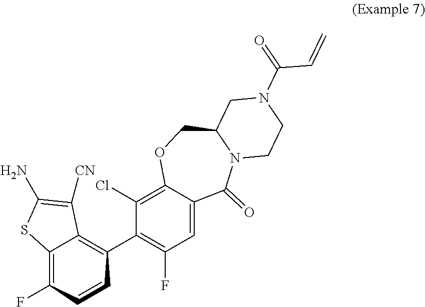

Acryloyl chloride (0.0022 mL, 0.027 mmol) was added to an ice-cooled mixture of 4-[(4aR)-7-chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, P atropisomer (0.013 g, 0.027 mmol) and potassium carbonate (0.011 g, 0.082 mmol) in EtOAc (0.27 mL) and water (0.27 mL). After 20 minutes, diluted with EtOAc and brine. The organic extract was dried over sodium sulfate, filtered, and concentrated in vacuo. The crude was purified by silica gel flash chromatography (20-100% acetone in hexanes) to give the title compound (0.011 g, 78%). ES/MS m/z (³⁵Cl/³⁷Cl) 515/517 [M+H]⁺.

### Example 8

### 4-[(4aR)-7-Chloro-9-fluoro-11-oxo-3-prop-2-enoyl-2,4,4a,5-tetrahydro-1H-pyrazino[2,1-c][1,4]benzoxazepin-8-yl]-2-amino-7-fluoro-benzothiophene-3-carbonitrile, M atropisomer

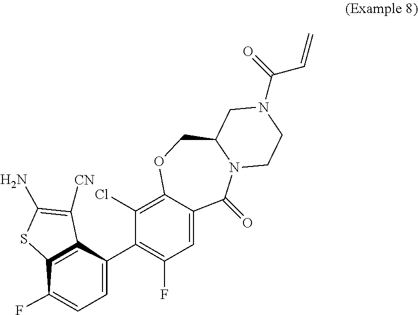

### Example 8

Acryloyl chloride (0.25M in DCM, 1.1 mL, 0.28 mmol) was added to an ice-cooled mixture of 4-[(4aR)-7-chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile, M atropisomer (0.128 g, 0.28 mmol) and potassium carbonate (0.12 g, 0.83 mmol) in EtOAc (3 mL) and water (3 mL). After five minutes, diluted with EtOAc and separated layers. The organic extract was dried over magnesium sulfate, filtered, and concentrated in vacuo. The crude was purified by silica gel flash chromatography (0-10% 7N ammoniated MeOH in DCM) to give the title compound (0.13 g, 87%). ES/MS m/z (³⁵Cl/³⁷Cl) 515/517 [M+H]⁺.

### Biological Assays

The following assays demonstrate that the exemplified compounds are inhibitors of KRas G12C and inhibit growth of certain tumors in vitro and/or in vivo.

### KRas G12C Probe Occupancy TR-FRET Assay

The purpose of this assay is to measure the ability of an inhibitor to compete with a probe for binding to and covalently modifying KRas G12C at codon 12. The signal is generated by the time-resolved transfer of fluorescence between europium on an antibody bound to KRas G12C Europium-labeled Anti-Histidine Tag Antibody LanthaScreen (the Eu Anti-His antibody) and fluorescent Tracer 647 (Alexa Fluor™) bound to KRas G12C through streptavidin and a biotinylated inhibitor (the “KRas Probe”, see Preparation 223).

Inhibitors are tested in dose response format from 10 mM stocks in 100% DMSO. The Labycyte Echo® 555 is used to dilute and transfer 100 nL per well containing a 10 point, 2.8-fold serial dilution to an assay plate. Two copies of the assay plate are prepared to measure the potency after 5 and 60 minutes incubation of the inhibitor with KRas G12C. His-tagged KRas G12C (20 nM) is added to the plates in assay buffer (20 mM Tris-HCl, pH 7.5, 0.01% TX-100, and 1 mM DTT). After 5 or 60 minutes incubation, 1 μM KRas Probe is added and allowed to covalently modify free KRas G12C for 1 hour. This is diluted 4-fold in buffer containing Eu Anti-His antibody and Streptavidin-Coated Tracer 647 (both from Life Technologies) to achieve KRas G12C (5 nM), Anti-His Antibody (2 nM), KRas Probe (300 nM), and Streptavidin Coated Tracer 647 (500 nM). After 30 minutes, the fluorescent signal is read on an Envision™ Plate Reader (excitation at 340 nM, tracer emission (em) at 665 nM, and antibody emission at 615 nM). Maximum control wells lack inhibitor and minimum control wells lack both inhibitor and KRas G12C. The signal ratio (em at 665/em at 615) is converted to percent inhibition using the following equation: % Inhibition=100−[(Test Compound Signal−Median Minimum Signal)/(Median Maximum Signal−Median Minimum Signal)×100]. The IC₅₀ is determined by fitting the percent inhibition at each inhibitor concentration to the four parameter nonlinear logistic equation using Genedata Screener®: y=(A+((B−A)/(1+((C/x)∧D)))) where, y=% inhibition, A=minimum asymptote, B=maximum asymptote, C=relative IC₅₀ or the inhibitor concentration producing 50% inhibition within the fitted range of both asymptotes, and D=Hill Slope. Compounds within the scope of this disclosure are evaluated in this assay substantially as described above. Exemplified compounds of the disclosure evaluated in this assay exhibit KRas G12C inhibitor activity by competing with a probe for binding to and covalently modifying KRas G12C at codon 12 as shown in Table 3.

TABLE 3
KRas G12C Probe Occupancy TR-FRET Assay

| Compound | IC₅₀ (nM) @ 5 min | IC₅₀ (nM) @ 60 min |
|---|---|---|
| Example 1 | 18600 | 16000 |
| Example 2 | <15 | <15 |
| Example 3 | 451 | 843 |
| Example 4 | <15 | <15 |
| Example 5 | 3270 | 3250 |
| Example 6 | <15 | <15 |
| Example 7 | 26900 | 17900 |
| Example 8 | <15 | <15 |

As illustrated in Table 3, Examples 2, 4, 6, and 8 (the M atropisomer designations) represent superior IC50 concentrations relative to Examples 1, 3, 5, and 7 (the P atropisomer designations).

### H358 Cellular Phospho-ERK AlphaLISA®

The purpose of this assay is to measure the ability of test compounds to inhibit the phosphorylation of p-ERK1/2, a downstream effector of KRas in human lung cancer cells H358 (ATCC CRL-5807). Briefly, the AlphaLISA® SureFire® Ultra™ p-ERK 1/2 (Thr202/Tyr204) assay is a sandwich immunoassay for quantitative detection of phospho-ERK 1/2 (phosphorylated on Thr202/Tyr204 in ERK1, or Thr185/Tyr187 in ERK2) in cellular lysates using Alpha Technology (Perkin Elmer Cat #ALSU-PERK-A50K).

H358 cells are plated at 40K cells per well in 100 μL media (RPMI 1640, GIBCO Cat #22400-071) containing 10% FBS (GIBCO Cat #: 10082-147) in a 96 well plate (Costar #3596) and are incubated overnight in humid trays at 37° C., 5% CO₂. The next morning, 10 μL of serially-diluted (3-fold) test compounds (50 μM top concentration) and 10 μL of controls (Maximum signal wells: 5% DMSO and Minimum signal wells: 2 μM of N-(3-{3-cyclopropyl-5-[(2-fluoro-4-iodophenyl)amino]-6,8-dimethyl-2,4,7-trioxo-3,4,6,7-tetrahydropyrido[4,3-D]pyrimidin-1(2H)-yl}phenyl)acetamide (trametinib, as a positive control) are added to the cell plate and incubated for 2 hours in humid trays at 37° C./5% CO₂. Lysis Buffer is prepared at ambient temperature containing a protease and phosphatase inhibitor cocktail. Culture medium is removed by inverting and shaking the cell plate in the sink and then blotting onto a paper towel. Lysis buffer is added to the cell plate (50 μL per well) and the plate is incubated at ambient temperature for 10 minutes on a shaker. For p-ERK detection, acceptor beads are diluted into a suspension mixture with buffer. Using a STARlet liquid handler, 5 μL of acceptor beads and 2 μL of cell lysate are transferred as a single-step in-tip dilution to a 384 well assay plate. The assay plate is sealed with foil and is incubated at ambient temperature for 2 hours. Donor beads are diluted into a suspension mixture with buffer. Using the STARlet, 5 μL of donor beads are added to the assay plate that is then sealed, wrapped with foil. The plate is incubated at ambient temperature for 2 hours in the dark. The assay plate is then read on an EnVision™ Plate Reader (Perkin Elmer) using a luminescence program.

The signal is converted to percent inhibition using the following equation: % Inhibition=100−[(Test Compound Signal−Median Minimum Signal)/(Median Maximum Signal−Median Min Signal)×100]. The Maximum signal is a control well without inhibitor. The Minimum signal is a control well containing a reference inhibitor sufficient to fully inhibit activity. The IC₅₀ is determined by fitting the percent inhibition at each inhibitor concentration to the four parameter nonlinear logistic equation using Genedata Screener®: y=(A+((B−A)/(1+((C/x)∧D)))) where, y=% inhibition, A=minimum asymptote, B=maximum asymptote, C=relative IC₅₀ or the inhibitor concentration producing 50% inhibition within the fitted range of both asymptotes, and D=Hill Slope.

Compounds within the scope of this disclosure are evaluated in this assay substantially as described above. The compounds of the Examples exhibit an ability to inhibit the phosphorylation of p-ERK1/2. Data in Table 4 show that the compounds of the Examples exhibit KRas G12C inhibition activity in this cellular assay.

TABLE 4
H358 Cellular Phospho-ERK AlphaLISA ®

| Compound | IC₅₀ (nM) |
|---|---|
| Example 1 | 2460 |
| Example 2 | 1.78 |
| Example 3 | 206 |
| Example 4 | 1.31 |
| Example 5 | 330 |
| Example 6 | 0.649 |
| Example 7 | 2980 |
| Example 8 | 0.902 |

### H358 Cellular Active RAS GTPase ELISA

The purpose of this assay is to measure the ability of test compounds to inhibit constitutive RAS GTPase activity in human lung cancer cells H358 (ATCC CRL-5807). The RAS GTPase ELISA kit (Active Motif Cat #52097) contains a 96-well plate pre-coated with glutathione in order to capture a kit-supplied GST-Raf-RBD protein. Activated RAS (GTP-bound) in cell extracts specifically bind to the Raf-RBD. Bound RAS is detected with a primary antibody that recognizes human KRas. A secondary antibody conjugated with HRP recognizes the primary antibody and a development solution provides a chemiluminescent readout.

H358 cells are plated at 80,000 cells/well in 90 μL serum free media (RPMI 1640, GIBCO) and incubated overnight at 37° C./5% CO₂. The next morning, 10 μL of serially-diluted (3-fold) test compounds (500 μM top concentration) and 10 μL of controls (Maximum signal wells: 5% DMSO and Minimum signal wells: 500 μM 1-[4-[6-chloro-8-fluoro-7-(3-hydroxy-1-naphthyl)quinazolin-4-yl]piperazin-1-yl]prop-2-en-1-one, WO2015054572 as an inhibitor) are added to the cell plate and incubated for 2 hours at 37° C./5% CO₂. Complete Lysis/Binding Buffer is prepared containing Protease Inhibitor cocktail and GST-Raf-RBD and stored on ice. One hour before cell plate incubation is completed, 50 μL of GST-Raf-RBD is diluted in lysis/binding buffer, and buffer is added to the ELISA assay plate and which is incubated for 1 hour at 4° C. with gently rocking. After 2 hours, the cells are washed with 100 μL ice-cold PBS and lysed with 100 μL lysis/binding buffer. The cell plate is shaken for 10 minutes at ambient temperature. The cell plate is then centrifuged at 1500 rpm for 10 minutes at ambient temperature. During this time, 1×Wash Buffer is prepared at ambient temperature and then is used to wash (3×100 μL) the GST-Raf-RBD coated assay plate. After washing, 50 μL of cell lysate is added to the GST-Raf-RBD coated assay plate and incubated for 1 hour at ambient temperature with gentle shaking. During this incubation period, 1×Antibody Binding Buffer is prepared and brought to ambient temperature. The assay plate is washed 3×100 μL with 1×Wash Buffer and then 50 μL of Primary Antibody (diluted 1:500 in 1×Antibody Binding buffer) is added. The plate is incubated for 1 hour at ambient temperature. The assay plate is washed 3×100 μL with 1×Wash Buffer and then 50 μL of Secondary Antibody (diluted 1:5000 in 1×Antibody Binding buffer) is added and incubated for 1 hour at ambient temperature. The assay plate is washed 4×100 μL with 1×Wash buffer and then 50 μL of chemiluminescent working solution is added at ambient temperature. The assay plate is then read on an EnVision™ Plate Reader (Perkin Elmer) using a luminescence program.

The signal is converted to percent inhibition using the following equation: % Inhibition=100−[(Test Compound Signal−Median Minimum Signal)/(Median Maximum Signal−Median Minimum Signal)×100]. The Maximum signal is a control well without inhibitor. The Minimum signal is a control well containing a reference inhibitor sufficient to fully inhibit activity. The IC₅₀ is determined by fitting the percent inhibition at each inhibitor concentration to the four parameter nonlinear logistic equation using Genedata Screener®: y=(A+((B−A)/(1+((C/x)∧D)))) where, y=% inhibition, A=minimum asymptote, B=maximum asymptote, C=relative IC₅₀ or the inhibitor concentration producing 50% inhibition within the fitted range of both asymptotes, and D=Hill Slope.

Compounds within the scope of this disclosure are evaluated in this assay substantially as described above. The compounds of the Examples exhibit an ability to inhibit constitutive RAS GTPase activity. Data in Table 5 show that the compounds of the Examples exhibit KRas-GTP inhibition activity in this human lung cancer cell culture.

TABLE 5
H358 Cellular Active RAS GTPase ELISA

| Compound | IC₅₀ (nM) |
|---|---|
| Example 1 | 14400 |
| Example 2 | 6.71 |
| Example 3 | 902 |
| Example 4 | 5.66 |
| Example 5 | 1770 |
| Example 6 | 3.31 |
| Example 7 | 23800 |
| Example 8 | 4.62 |

### Additional Embodiments

Embodiment 1. A compound selected from the group consisting of:

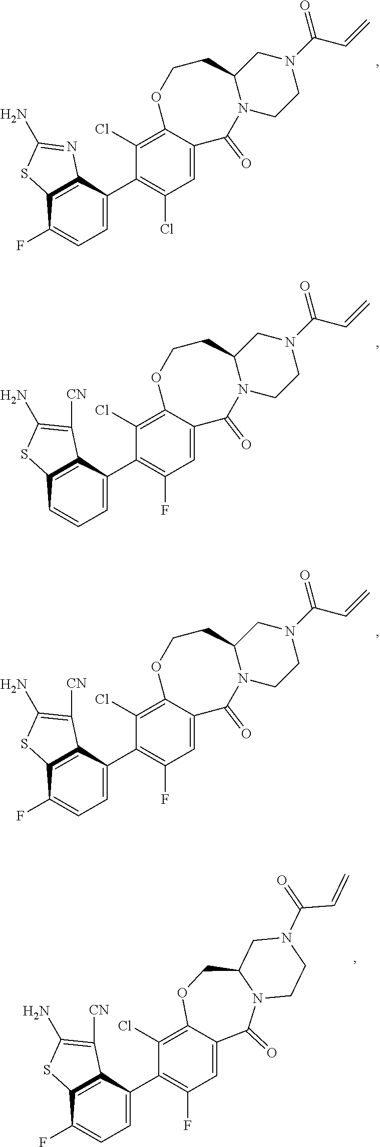

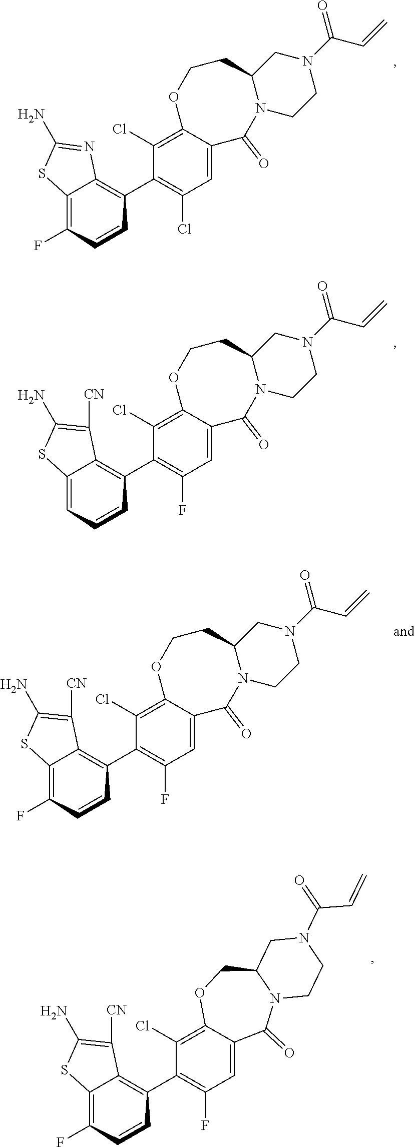

or a pharmaceutically acceptable salt thereof.
Embodiment 2. The compound of embodiment 1 selected from the group consisting of:

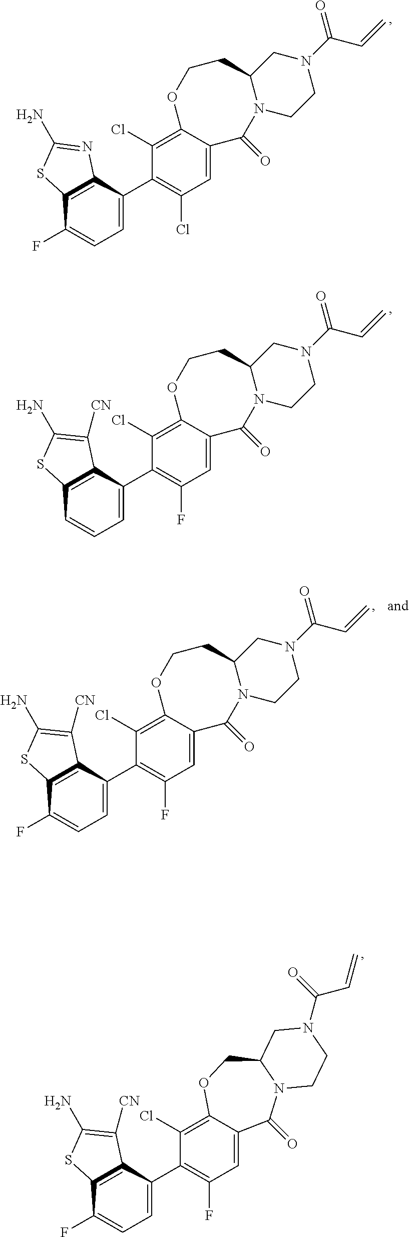

or a pharmaceutically acceptable salt thereof.
Embodiment 3. The compound of embodiment 1 selected from the group consisting of:

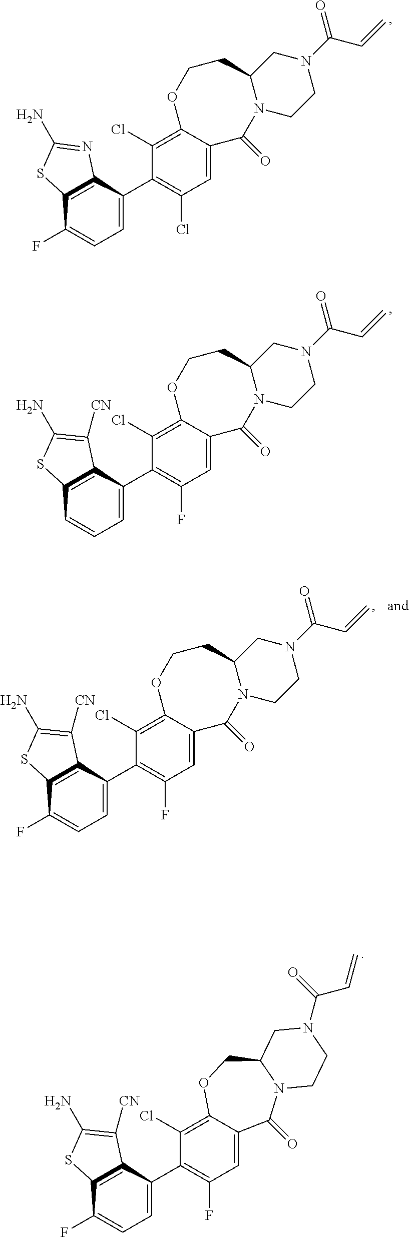

Embodiment 4. The compound of embodiment 1 selected from the group consisting of:

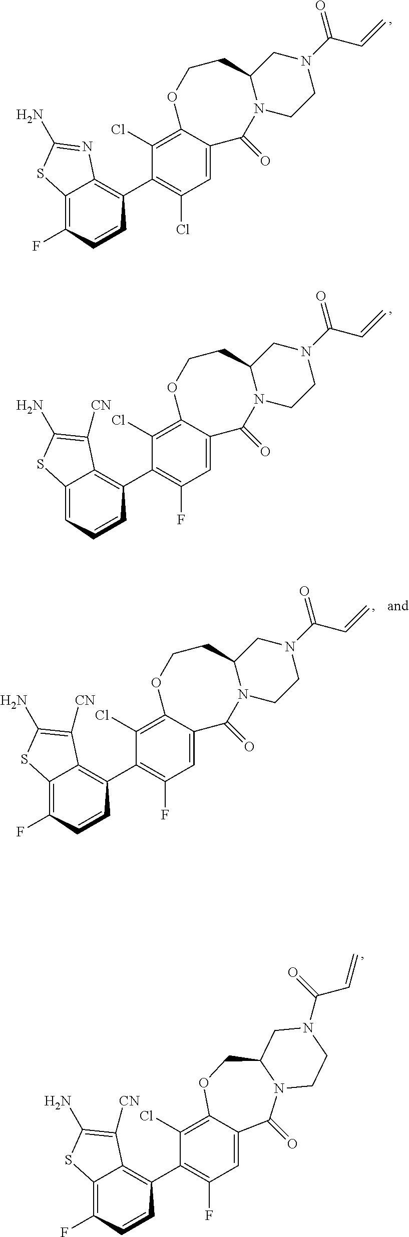

or a pharmaceutically acceptable salt thereof.
Embodiment 5. The compound of embodiment 1 selected from the group consisting of:

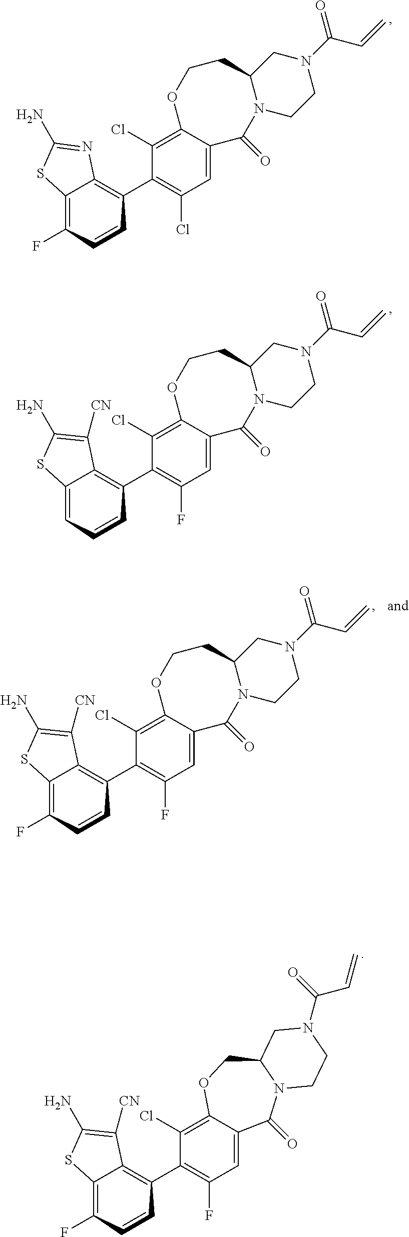

Embodiment 6. The compound of embodiment 1 selected from the group consisting of:

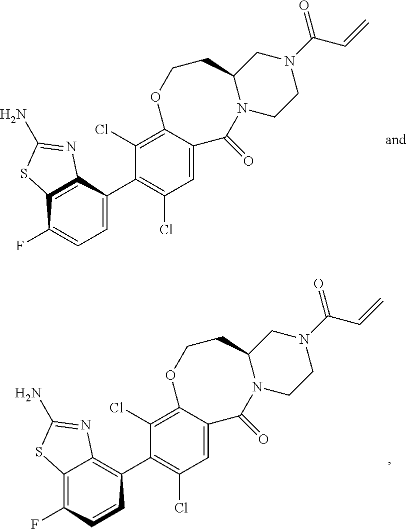

or a pharmaceutically acceptable salt thereof.
Embodiment 7. The compound of embodiments 1 or 6 wherein the compound is

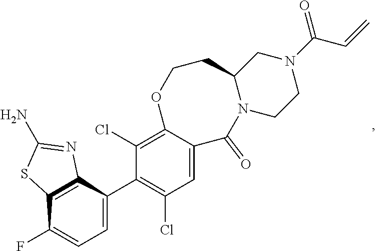

or a pharmaceutically acceptable salt thereof.
Embodiment 8. The compound of embodiment s 1 or 6 wherein the compound is

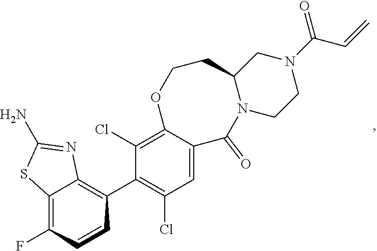

or a pharmaceutically acceptable salt thereof.
Embodiment 9. The compound of embodiment 1 selected from the group consisting of:

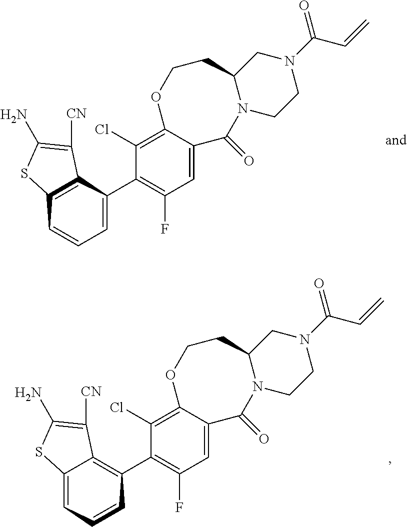

or a pharmaceutically acceptable salt thereof.
Embodiment 10. The compound of embodiments 1 or 9 wherein the compound is

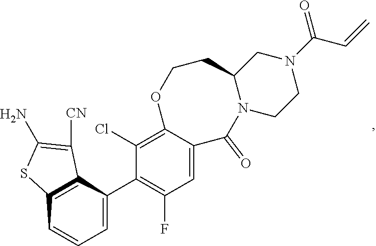

or a pharmaceutically acceptable salt thereof.
Embodiment 11. The compound of embodiments 1 or 9 wherein the compound is

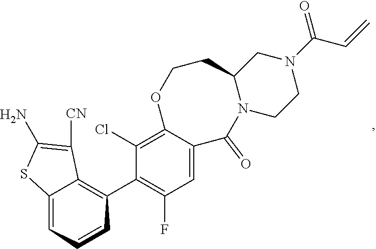

or a pharmaceutically acceptable salt thereof.
Embodiment 12. The compound of embodiment 1 selected from the group consisting of:

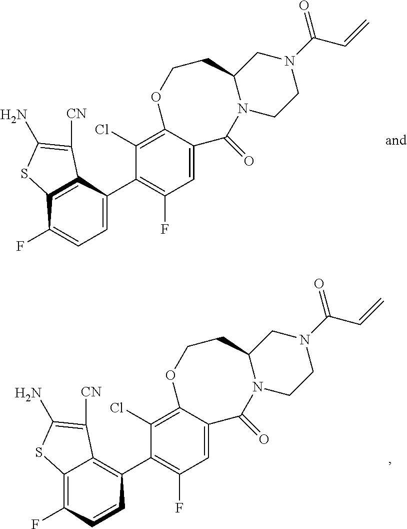

or a pharmaceutically acceptable salt thereof.
Embodiment 13. The compound of embodiments 1 or 12 wherein the compound is

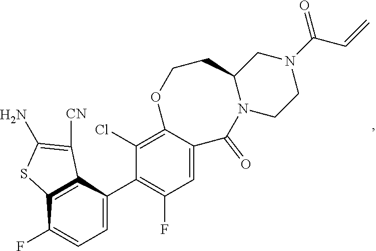

or a pharmaceutically acceptable salt thereof.
Embodiment 14. The compound of embodiments 1 or 12 wherein the compound is

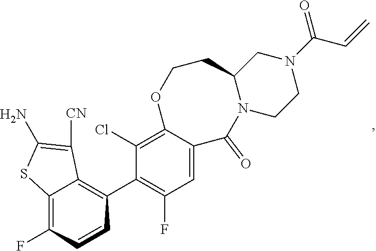

or a pharmaceutically acceptable salt thereof.
Embodiment 15. The compound of embodiment 1 selected from the group consisting of:

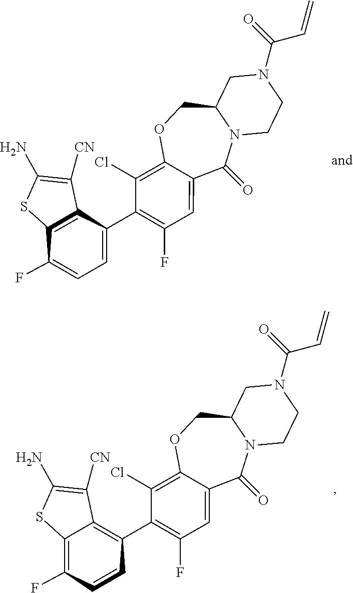

or a pharmaceutically acceptable salt thereof.
Embodiment 16. The compound of embodiments 1 or 15 wherein the compound is

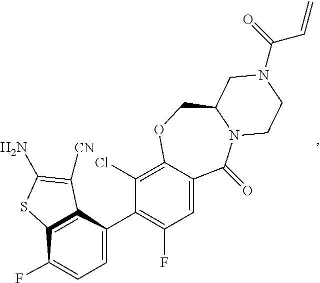

or a pharmaceutically acceptable salt thereof.
Embodiment 17. The compound of embodiments 1 or 15 wherein the compound is

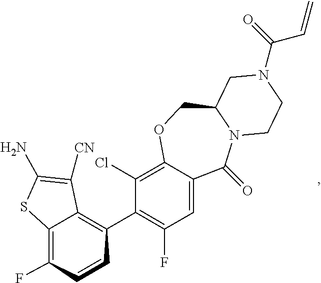

or a pharmaceutically acceptable salt thereof.
Embodiment 18. A pharmaceutical composition comprising a compound according to any one of embodiments 1-17, or a pharmaceutically acceptable salt thereof, and a pharmaceutically acceptable carrier, diluent or excipient.
Embodiment 19. A method of treating a patient for cancer wherein one or more cells express KRas G12C mutant protein, comprising administering to a patient in need thereof, an effective amount of a pharmaceutical composition according to embodiment 6, wherein the cancer is selected from lung cancer, advanced non-small cell lung cancer, pancreatic cancer, cervical cancer, esophageal cancer, endometrial cancer, ovarian cancer, cholangiocarcinoma, and colorectal cancer.
Embodiment 20. A method of treating a patient for cancer wherein one or more cells express KRas G12C mutant protein, comprising administering to a patient in need thereof, an effective amount of a Compound according to any one of embodiments 1-17, or a pharmaceutically acceptable salt thereof, wherein the cancer is selected from lung cancer, advanced non-small cell lung cancer, pancreatic cancer, cervical cancer, esophageal cancer, endometrial cancer, ovarian cancer, cholangiocarcinoma, and colorectal cancer.
Embodiment 21. The method according to embodiment 19 wherein the cancer is non-small cell lung cancer.
Embodiment 22. The method according to embodiment 19 or 20 wherein the cancer is advanced non-small cell lung cancer.
Embodiment 23. The method according to embodiment 19 wherein the cancer is colorectal cancer.
Embodiment 24. The method according to embodiment 19 wherein the cancer is pancreatic cancer.
Embodiment 25. The method according to embodiment 19 wherein the patient has a cancer that was determined to have one or more cells expressing the KRas G12C mutant protein prior to administration of the compound or a pharmaceutically acceptable salt thereof.
Embodiment 26. A method of treating a patient with a cancer that has a KRAS G12C mutation comprising administering to the patient in need thereof an effective amount of a compound according to any one of embodiments 1 to 17, or a pharmaceutically acceptable salt thereof.
Embodiment 27. The method according to any one of embodiments 19 to 25, wherein the patient is also administered an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.
Embodiment 28. The method according to any one of embodiments 19 to 26, wherein the patient has not received prior therapy with a KRAS G12C inhibitor, or a pharmaceutically acceptable salt thereof.
Embodiment 29. The method according to any one of embodiments 19 to 27, wherein the patient has not received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.
Embodiment 30. The method according to any one of embodiments 19 to 27, wherein the patient received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.
Embodiment 31. The method according to any one of embodiments 19 to 26, wherein the patient received prior therapy with a KRAS G12C inhibitor, or a pharmaceutically acceptable salt thereof.
Embodiment 32. The method according to any one of embodiments 19 to 26 or 30, wherein the patient has not received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.
Embodiment 33. The method according to any one of embodiments 19 to 26 or 30, wherein the patient received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.
Embodiment 34. The compound, or a pharmaceutically acceptable salt thereof, according to any one of embodiments 1 to 17, for use in therapy.
Embodiment 35. The compound, or a pharmaceutically acceptable salt thereof, according to any one of embodiments 1 to 17, for use in the treatment of cancer.
Embodiment 36. The compound, or a pharmaceutically acceptable salt thereof, for use according to embodiment 35, wherein the cancer is selected from lung cancer, advanced non-small cell lung cancer, pancreatic cancer, cervical cancer, esophageal cancer, endometrial cancer, ovarian cancer, cholangiocarcinoma, and colorectal cancer.
Embodiment 37. The compound, or a pharmaceutically acceptable salt thereof, according to any one of embodiments 1 to 17 for use in simultaneous, separate or sequential combination with one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof, in the treatment of cancer.
Embodiment 38. A method of preparation of a compound according to embodiments 1 or 9 comprising:
   separating M and P atropisomers of tert-Butyl (13aS)-9-[2-(tert-butoxycarbonylamino)-3-cyano-benzothiophen-4-yl]-10-chloro-8-fluoro-6-oxo-1,3,4,12,13,13a-hexahydropyrazino[2,1-d][1,5]benzoxazocine-2-carboxylate by silica gel flash chromatography.
Embodiment 39. The method of embodiment 38, further comprising the use of 0-30% acetone in hexanes.
Embodiment 40. The method of embodiments 38 or 39 further comprising the steps of:
   adding TFA to a solution of P atropisomer of tert-butyl (13aS)-9-[2-(tert-butoxycarbonylamino)-3-cyano-benzothiophen-4-yl]-10-chloro-8-fluoro-6-oxo-1,3,4,12,13,13a-hexahydropyrazino[2,1-d][1,5]benzoxazocine-2-carboxylatein DCM, followed by
   stirring at about room temperature for about three hours, and concentrating in vacuo.
Embodiment 41. The method of any one of embodiments 38 to 40 further comprising:
   purifying a P atropisomer of 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile by silica gel flash chromatography.
Embodiment 42. The method of embodiment 41, further comprising the use of 4-10% 7N ammoniated MeOH in DCM.
Embodiment 43. The method of any one of embodiments 38 to 42 further comprising the steps of:
   cooling to about −78° C. a solution of P atropisomer of 4-[(13aS)-10-Chloro-8-fluoro-6-oxo-2,3,4,12,13,13a-hexahydro-1H-pyrazino[2,1-d][1,5]benzoxazocin-9-yl]-2-amino-benzothiophene-3-carbonitrile, atropisomer 1 and DIEA in DCM, followed by
   adding acryloyl chloride, followed about five minutes by,
   diluting with a small amount of 2-propanol, and concentrating in vacuo.
Embodiment 44. The method of any one of embodiments 38 to 43 further comprising the steps of:
   purifying a P atropisomer of 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-benzothiophene-3-carbonitrile by silica gel flash chromatography.
Embodiment 45. The method of any one of embodiments 38 to 44, further comprising the use of 20-100% acetone in hexanes.
Embodiment 46. A method of preparation of a compound according to embodiments 1 or 12 comprising:
   reacting tert-butyl (13aS)-9-bromo-10-chloro-8-fluoro-6-oxo-1,3,4,12,13,13a-hexahydropyrazino[2,1-d][1,5]benzoxazocine-2-carboxylate, tert-butyl N-[3-cyano-4-(5,5-dimethyl-1,3,2-dioxaborinan-2-yl)-7-fluoro-benzothiophen-2-yl]carbamate, potassium carbonate, and dichloro[(S)-(−)-2,2′-bis(diphenylphosphino)-1,1′-binaphthyl]palladium(II) to 1,4-dioxane in a reaction flask.
Embodiment 47. The method of embodiment 46, wherein 1,4-dioxane had been flushed with nitrogen by direct sparge for 30 minutes.
Embodiment 48. The method of embodiments 46 or 47, further comprising the steps of:
   sealing and heating at 105° C. the reaction flask for about 14 hours, followed by
   adding additional tert-butyl N-[3-cyano-4-(5,5-dimethyl-1,3,2-dioxaborinan-2-yl)-7-fluoro-benzothiophen-2-yl]carbamate and dichloro[(S)-(−)-2,2′-bis(diphenylphosphino)-1,1′-binaphthyl]palladium(II), followed by
   sealing and heating at 105° C. the reaction flask for about 14 hours to produce a reaction mixture.
Embodiment 49. The method of embodiment 48, further comprising the steps of: filtering the reaction mixture through diatomaceous earth, and rinsing with EtOAc to produce a filtrate.
Embodiment 50. The method of embodiment 49, further comprising the steps of: concentrating the filtrate in vacuo, and diluting with EtOAc, water, and brine to produce an aqueous layer.
Embodiment 51. The method of any one of embodiments 46 to 50, further comprising the steps of:
   extracting the aqueous layer with EtOAc to produce at least one organic extract, followed by
   drying the organic extracts over magnesium sulfate, filtering, and concentrating in vacuo.
Embodiment 52. The method of any one of embodiments 46 to 51 further comprising the steps of:
   purifying M and P atropisomers of tert-Butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate by silica gel flash chromatography.
Embodiment 53. The method of embodiment 52, further comprising the use of 10-40% acetone in hexanes.
Embodiment 54. The method of any one of embodiments 46 to 53 further comprising the steps of:
   adding TFA to a solution of P atropisomer of tert-butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate in DCM, and
   stirring at about room temperature for about 30 minutes, followed by
   concentrating in vacuo to give a crude deprotected residue.
Embodiment 55. The method of embodiment 54 further comprising the steps of:
   dissolving the crude deprotected residue in DCM and DIEA, followed by cooling to −78° C., followed by
   adding acryloyl chloride in DCM, followed about 30 minutes by concentrating in vacuo.
Embodiment 56. The method of any one of embodiments 46 to 55 further comprising the steps of:
   purifying P atropisomer of 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile by silica gel flash chromatography.
Embodiment 57. The method of embodiment 56 further comprising the use of 20-80% acetone in hexanes.
Embodiment 58. The method of any one of embodiments 46 to 57 further comprising the steps of:
   bubbling HCl gas for about five minutes into an ice-cooled solution of M atropisomer of tert-butyl (13aS)-9-[2-(tert-butoxycarbonylamino)-3-cyano-7-fluoro-benzothiophen-4-yl]-10-chloro-8-fluoro-6-oxo-1,3,4,12,13,13a-hexahydropyrazino[2,1-d][1,5]benzoxazocine-2-carboxylate in DCM and 2-propanol,
   stirring at about room temperature for about five hours, followed by
   cooling in an ice bath, followed by
   bubbling HCl gas for about five minutes, followed by
   stirring at about room temperature for about 14 hours, followed by
   concentrating in vacuo to produce a residue,
   twice diluting the residue with n-heptane, followed by
   concentrating in vacuo, followed by
   adding MTBE,
   stirring at about room temperature for about ten minutes, followed by
   filtering to produce a dihydrochloride salt.
Embodiment 59. The method of embodiment 58 further comprising the steps of: dissolving the dihydrochloride salt in water, followed by
   adding 2-Methyltetrahydrofuran and a solution of potassium carbonate in water, followed by
   vigorously stirring while being cooled in an ice bath, followed by
   dropwise adding acryloyl chloride in 2-methyltetrahydrofuran, followed about five minutes by,
   diluting with brine and 2-methyltetrahydrofuran,
   twice extracting the aqueous layer with 2-methyltetrahydrofuran, followed by
   washing the combined organic extracts with water, and
   drying over magnesium sulfate, filtering, and concentrating in vacuo.
Embodiment 60. The method of any one of embodiments 37 to 54 further comprising the steps of:
   purifying M atropisomer of 4-[(13aS)-10-Chloro-8-fluoro-6-oxo-2-prop-2-enoyl-1,3,4,12,13,13a-hexahydropyrazino[2,1-d][1,5]benzoxazocin-9-yl]-2-amino-7-fluoro-benzothiophene-3-carbonitrile by silica gel flash chromatography.
Embodiment 61. The method of 60 further comprising the use of 20-30% 4:1 EtOAc:MeOH in 3:1 EtOAc:hexanes.
Embodiment 62. The method of preparation of a compound according to embodiments 1 or 15 comprising the steps of:
   adding a solution of tert-butyl (4aR)-7-chloro-9-fluoro-8-iodo-11-oxo-2,4,4a,5-tetrahydro-1H-pyrazino[2,1-c][1,4]benzoxazepine-3-carboxylate, tert-butyl N-[3-cyano-7-fluoro-4-(4,4,5,5-tetramethyl-1,3,2-dioxaborolan-2-yl)benzothiophen-2-yl]carbamate, and potassium phosphate in water and 1,4-dioxane to a first reaction flask,
   flushing the first reaction flask with nitrogen by direct sparge for about 10 minutes,
   adding 1,1′-Bis(di-tert-butylphosphino)ferrocene palladium dichloride to the first reaction flask, and
   sealing and heating at about 70° C. the first reaction flask for about three hours to produce a first reaction mixture.
Embodiment 63. The method of preparation of a compound according to any one of embodiments 1, 15, or 62 comprising the steps of:
   adding a solution of tert-butyl (4aR)-7-chloro-9-fluoro-8-iodo-11-oxo-2,4,4a,5-tetrahydro-1H-pyrazino[2,1-c][1,4]benzoxazepine-3-carboxylate, tert-butyl N-[3-cyano-7-fluoro-4-(4,4,5,5-tetramethyl-1,3,2-dioxaborolan-2-yl)benzothiophen-2-yl]carbamate, and potassium phosphate in water and 1,4-dioxane to a second reaction flask,
   flushing the second reaction flask with nitrogen by direct sparge for about 10 minutes,
   adding 1,1′-Bis(di-tert-butylphosphino)ferrocene palladium dichloride to the second reaction flask, and
   sealing and heating at about 70° C. the second reaction flask for about five hours to produce a second reaction mixture.
Embodiment 64. The method of preparation of a compound according to embodiment 63, further comprising the steps of:
   combining the first and second reaction mixtures to produce a combined reaction mixture,
   filtering the combined reaction mixture through diatomaceous earth, and
   rinsing with EtOAc to produce a filtrate.
Embodiment 65. The method of preparation of a compound according to embodiment 64, further comprising the steps of:
   diluting the filtrate with MTBE and saturated aqueous sodium bicarbonate to produce an organic extract, followed by
   washing the organic extract with brine,
   drying over magnesium sulfate, filtering, and concentrating in vacuo to produce a crude.
Embodiment 66. The method of preparation of a compound according to embodiment 65, further comprising the steps of:
   purifying M and P atropisomers of tert-Butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate by silica gel flash chromatography.
Embodiment 67. The method of preparation of a compound according to embodiment 66, further comprising the use of 0-30% acetone in hexanes.
Embodiment 68. The method of preparation of a compound according to embodiments 66 or 67, further comprising the steps of:
   further purifying P atropisomers of tert-Butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate by silica gel flash chromatography.
Embodiment 69. The method of preparation of a compound according to embodiment 68, further comprising the use of 0-100% EtOAc in hexanes.
Embodiment 70. The method of preparation of a compound according to any one of embodiments 66 to 69, further comprising the steps of:
   adding TFA to a solution of P atropisomer tert-butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate in DCM, followed by
   stirring at room temperature for about 30 minutes,
   concentrating in vacuo, and
   purifying P atropisomer of 4-[(4aR)-7-chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile by silica gel flash chromatography.
Embodiment 71. The method of preparation of a compound according to embodiment 70, further comprising use of 0-10% MeOH in DCM, followed by 0-10% 7N ammoniated MeOH in DCM.
Embodiment 72. The method of preparation of a compound according to any one of embodiments 66 to 71, further comprising the steps of:
   adding acryloyl chloride to an ice-cooled mixture of P atropisomer of 4-[(4aR)-7-chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile and potassium carbonate in EtOAc and water, followed about 20 minutes later by
   diluting with EtOAc and brine,
   drying the resulting the organic extract over sodium sulfate, filtering, and concentrating in vacuo.
Embodiment 73. The method of preparation of a compound according to any one of embodiments 66 to 72, further comprising:
   purifying P atropisomer of 4-[(4aR)-7-Chloro-9-fluoro-11-oxo-3-prop-2-enoyl-2,4,4a,5-tetrahydro-1H-pyrazino[2,1-c][1,4]benzoxazepin-8-yl]-2-amino-7-fluoro-benzothiophene-3-carbonitrile by silica gel flash chromatography.
Embodiment 74. The method of preparation of a compound according to embodiment 73, further comprising use of 20-100% acetone in hexanes.
Embodiment 75. The method of preparation of a compound according to any one of embodiments 66 to 74, further comprising the steps of:
   adding TFA to an ice-cooled solution of M atropisomer of tert-butyl (4aR)-8-[2-(tert-butoxycarbonylamino)-3-cyano-7-fluoro-benzothiophen-4-yl]-7-chloro-9-fluoro-11-oxo-2,4,4a,5-tetrahydro-1H-pyrazino[2,1-c][1,4]benzoxazepine-3-carboxylate in DCM,
   stirring at room temperature for about two hours, and
   concentrating in vacuo.
Embodiment 76. The method of preparation of a compound according to any one of embodiments 66 to 75, further comprising the steps of:
   purifying the M atropisomer of 4-[(4aR)-7-Chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile by silica gel flash chromatography.
Embodiment 77. The method of preparation of a compound according to embodiment 76, further comprising use of 0-10% 7N ammoniated MeOH in DCM.
Embodiment 78. The method of preparation of a compound according to any one of embodiments 66 to 77, further comprising the steps of:
   adding acryloyl chloride to an ice-cooled mixture of M atropisomer of 4-[(4aR)-7-chloro-9-fluoro-11-oxo-1,2,3,4,4a,5-hexahydropyrazino[2,1-c][1,4]benzoxazepin-8-yl]-2-amino-7-fluoro-benzothiophene-3-carbonitrile and potassium carbonate in EtOAc and water, followed about five minutes by,
   diluting with EtOAc,
   separating organic and aqueous layers, and
   drying the organic layer over magnesium sulfate, filtering, and concentrating in vacuo.
Embodiment 79. The method of preparation of a compound according to any one of embodiments 66 to 78, further comprising the steps of:
   purifying an M atropisomer of 4-[(4aR)-7-Chloro-9-fluoro-11-oxo-3-prop-2-enoyl-2,4,4a,5-tetrahydro-1H-pyrazino[2,1-c][1,4]benzoxazepin-8-yl]-2-amino-7-fluoro-benzothiophene-3-carbonitrile by silica gel flash chromatography.
Embodiment 80. The method of preparation of a compound according to embodiment 79, further comprising use of 0-10% 7N ammoniated MeOH in DCM.
Embodiment 81. A compound of the formula:

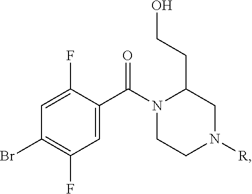

or a pharmaceutically acceptable salt thereof, wherein:
R is a protecting group.
Embodiment 82. The compound of embodiment 81 wherein R is Boc.
Embodiment 83. The compound of embodiments 81 or 82, wherein the compound of the formula is

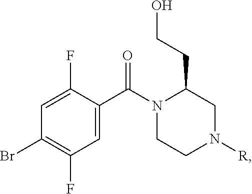

or a pharmaceutically acceptable salt thereof.
Embodiment 84. The compound of any one of embodiments 81 to 83, wherein the compound of the formula is

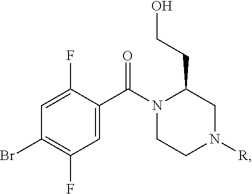

Embodiment 85. A compound of the formula:

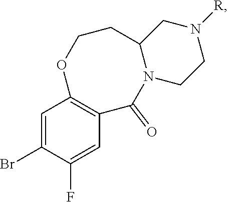

or a pharmaceutically acceptable salt thereof, wherein:
R is a protecting group.
Embodiment 86. The compound of embodiment 85 wherein R is Boc.
Embodiment 87. The compound of embodiments 85 or 86, wherein the compound

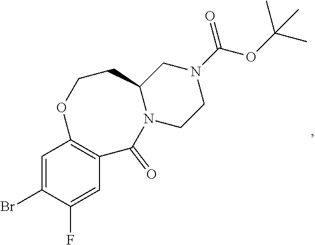 [SMILES: CC(C)(C)OC(=O)N1CCN2C(=O)c3cc(F)c(Br)cc3OCC[C@H]2C1]

or a pharmaceutically acceptable salt thereof.
Embodiment 88. The compound of any one of embodiments 85 to 87, wherein the compound is

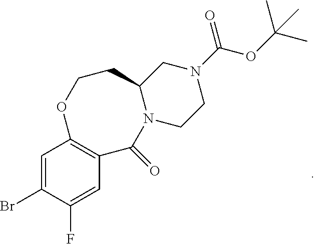 [SMILES: CC(C)(C)OC(=O)N1CCN2C(=O)c3cc(F)c(Br)cc3OCC[C@H]2C1]

Embodiment 89. A method of preparation of a compound according to any one of embodiments 81 to 88 comprising:
   combining tert-butyl (3S)-4-(4-bromo-2,5-difluoro-benzoyl)-3-(2-hydroxyethyl)piperazine-1-carboxylate with a cyclization base to give tert-butyl (13aS)-9-bromo-8-fluoro-6-oxo-1,3,4,12,13,13a-hexahydropyrazino[2,1-d][1,5]benzoxazocine-2-carboxylate.
Embodiment 90. The method of preparation of a compound according to embodiment 89 wherein the cyclization base is selected from the group consisting of sodium hydride, N,N-diisopropylethylamine (DIPEA), triethylamine (TEA), cesium carbonate, diazabicycloundecene (DBU), sodium tert-butoxide, sodium tert-pentoxide, sodium tert-amylate, potassium tert-pentoxide, and potassium tert-butoxide.
Embodiment 91. The method of preparation of a compound according to embodiments 89 or 90 wherein the cyclization base is selected from the group consisting of potassium tert-butoxide, sodium tert-amylate, potassium tert-pentoxide, sodium tert-butoxide, and sodium tert-pentoxide.
Embodiment 92. The method of preparation of a compound according to any one of embodiments 89 to 91 further comprising use of a cyclization solvent.
Embodiment 93. The method of preparation of a compound according to embodiment 92 wherein the cyclization solvent is selected from the group consisting of DMF, NMP, DMAc, DMSO, and THF.
Embodiment 94. A method of preparation of a compound according to any one of embodiments 89 to 93 further comprising:
   providing sodium tert-pentoxide to a polar aprotic solvent,
   stirring to form a reactor solution, and
   cooling the reactor solution to at least about 0° C.
Embodiment 95. The method of preparation of a compound according to embodiment 94 wherein the polar aprotic solvent is selected from the group consisting of DMAc, NMP, DMSO, and DMF.
Embodiment 96. The method of preparation of a compound according to embodiments 94 or 95 wherein the polar aprotic solvent is DMF.
Embodiment 97. A method of preparation of a compound further comprising: providing tert-butyl (3S)-4-(4-bromo-2,5-difluoro-benzoyl)-3-(2-hydroxyethyl)piperazine-1-carboxylate to the reactor solution over at least about 4 hours at about 0° C.
Embodiment 98. The method of preparation of a compound according to any one of embodiments 94 to 97 further comprising:
   adding a portion of the reactor solution including tert-butyl (3S)-4-(4-bromo-2,5-difluoro-benzoyl)-3-(2-hydroxyethyl)piperazine-1-carboxylate to water over at least about 1 hour within the range of about 10° C. to about 40° C. to form a preparation mixture,
   stirring the preparation mixture for at least about 32 hours within the range of about 10° C. to about 40° C.
Embodiment 99. The method of preparation of a compound according to embodiment 98 further comprising:
   isolating a solid from the preparation mixture by filtration and
   washing the solid from the preparation mixture with a combination of DMF and water.
Embodiment 100. The method of preparation of a compound according to embodiments 98 or 99 further comprising:
   adding the solid from the preparation mixture to EtOH to form a preparation solution.
Embodiment 101. The method of preparation of a compound according to any one of embodiments 98 to 100 further comprising:
   heating the preparation solution to at least about 30° C. and stirring for at least about 15 minutes.
Embodiment 102. The method of preparation of a compound according to any one of embodiments 98 to 101 further comprising:
   adding water to the preparation solution over at least about 2 hours.
Embodiment 103. The method of preparation of a compound according to any one of embodiments 98 to 102 further comprising:
   maintaining the preparation solution at about 30° C. for at least about 15 minutes.
Embodiment 104. The method of preparation of a compound according to any one of embodiments 98 to 103 further comprising:
   cooling the preparation solution to at least about 20° C. over at least about 2 hours.
Embodiment 105. The method of preparation of a compound according to any one of embodiments 98 to 104 further comprising:
   maintaining the preparation solution at about 20° C. for at least about 2 hours.
Embodiment 106. The method of preparation of a compound according to any one of embodiments 98 to 105 further comprising:
   isolating, washing, and drying a solid including tert-butyl (13aS)-9-bromo-8-fluoro-6-oxo-1,3,4,12,13,13a-hexahydropyrazino[2,1-d][1,5]benzoxazocine-2-carboxylate from the preparation solution.
Embodiment 107. The method of preparation of a compound according to embodiment 106 wherein the step of isolating the solid including tert-butyl (13aS)-9-bromo-8-fluoro-6-oxo-1,3,4,12,13,13a-hexahydropyrazino[2,1-d][1,5]benzoxazocine-2-carboxylate is by filtration.
Embodiment 108. The method of preparation of a compound according to embodiments 106 or 107 wherein the step of washing the solid including tert-butyl (13aS)-9-bromo-8-fluoro-6-oxo-1,3,4,12,13,13a-hexahydropyrazino[2,1-d][1,5]benzoxazocine-2-carboxylate is with a combination of EtOH and water.
Embodiment 109. The method of preparation of a compound according to any one of embodiments 106 to 108 wherein the step of drying the solid including tert-butyl (13aS)-9-bromo-8-fluoro-6-oxo-1,3,4,12,13,13a-hexahydropyrazino[2,1-d][1,5]benzoxazocine-2-carboxylate is under vacuum at about 50° C. for at least about 16 hours.
Embodiment 110. A method of preparation of a compound according to any one of embodiments 89 to 97 further comprising, prior to the steps of claim 89:
   adding CDMT with acetonitrile to a reactor and cooling the reactor to about 0° C., followed by
   adding an organic base to the reactor and stirring for at least 30 minutes at about 0° C., followed by
   adding 4-bromo-2,5-difluorobenzoic acid,

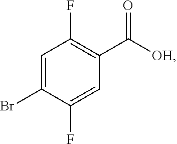 [SMILES: O=C(O)c1cc(F)c(Br)cc1F]

to the reactor and stirring for at least one hour at about 0° C. to form an acetonitrile coupling solution.
Embodiment 111. The method of preparation of a compound according to embodiment 110 further comprising:
   adding an inorganic base followed by water to a separate vessel, followed by
   adding tert-butyl (3S)-3-(2-hydroxyethyl)piperazine-1-carboxylate:phosphoric acid (1:1) and a base to the separate vessel to form an aqueous piperazine solution.
Embodiment 112. The method of preparation of a compound according to embodiment 110 wherein the organic base is NMM.
Embodiment 113. The method of preparation of a compound according to embodiment 111 wherein the inorganic base is K₂CO₃.
Embodiment 114. The method of preparation of a compound according to any one of embodiments 110 to 113 further comprising:
   adding the aqueous piperazine solution to the acetonitrile coupling solution of the reactor over at least 5 minutes while maintaining an internal temperature of about 0° C.
Embodiment 115. The method of preparation of a compound according to any one of embodiments 110 to 114 further comprising:
   stirring the solutions for at least 2.5 hours at about 0° C.
Embodiment 116. The method of preparation of a compound according to any one of embodiments 110 to 115 further comprising:
   removing the bottom layer of the solutions from the reactor,
   stirring the remaining solution for at least 64 hours at about 0° C.
Embodiment 117. The method of preparation of a compound according to any one of embodiments 110 to 116 further comprising:
   concentrating the remaining solution and adding 2-MeTHF with n-heptane.
Embodiment 118. The method of preparation of a compound according to any one of embodiments 110 to 117 further comprising:
   adding 1 M aq. NaHCO₃ to the remaining solution and stirring for at least 15 minutes.
Embodiment 119. The method of preparation of a compound according to any one of embodiments 110 to 118 further comprising:
   allowing the layers of the remaining solution to separate,
   removing the bottom layer of the remaining solution from the reactor, and
   repeating the steps of adding 1 M aq. NaHCO₃, stirring, and removing the bottom layer.
Embodiment 120. The method of preparation of a compound according to any one of embodiments 110 to 119 further comprising:
   adding 0.05 M aq. HCl to the remaining solution and stirring for at least 15 minutes.
Embodiment 121. The method of preparation of a compound according to any one of embodiments 110 to 120 further comprising:
   allowing the layers of the remaining solution to separate,
   removing the bottom layer of the remaining solution from the reactor.
Embodiment 122. The method of preparation of a compound according to any one of embodiments 110 to 121 further comprising:
   adding water to the remaining solution and stirring for at least 15 minutes.
Embodiment 123. The method of preparation of a compound according to any one of embodiments 110 to 122 further comprising:
   allowing the layers of the remaining solution to separate,
   removing the bottom layer of the remaining solution from the reactor.
Embodiment 124. The method of preparation of a compound according to any one of embodiments 110 to 123 further comprising:
   concentrating the remaining solution and adding 2-MeTHF, and
   repeating the steps of concentrating and adding 2-MeTHF.
Embodiment 125. The method of preparation of a compound according to any one of embodiments 110 to 124 further comprising:
   concentrating the remaining solution and adding DMF to provide tert-butyl (3S)-4-(4-bromo-2,5-difluoro-benzoyl)-3-(2-hydroxyethyl)piperazine-1-carboxylate.

What is claimed is:

1. A compound of the formula, 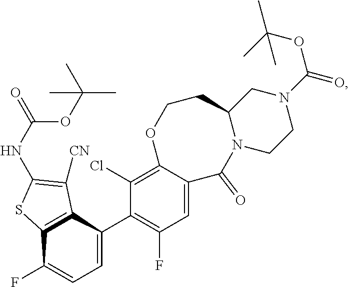
or a pharmaceutically acceptable salt thereof,
obtainable by combining tert-butyl (13aS)-9-bromo-10-chloro-8-fluoro-6-oxo-1,3,4,12,13,13a-hexahydropyrazino[2,1-d][1,5]benzoxazocine-2-carboxylate, tert-butyl N-[3-cyano-4-(5,5-dimethyl-1,3,2-dioxaborinan-2-yl)-7-fluoro-benzothiophen-2-yl]carbamate, potassium carbonate, and a catalyst in 1,4-dioxane, wherein the M:P atropisomer ratio is at least 4:1.

2. The compound of claim 1, further obtainable by adding additional tert-butyl N-[3-cyano-4-(5,5-dimethyl-1,3,2-dioxaborinan-2-yl)-7-fluoro-benzothiophen-2-yl]carbamate and the catalyst.

3. The compound of claim 1, wherein the catalyst comprises diacetate[(S)-(−)-2,2′-bis(diphenylphosphino)-1,1′-binaphthyl]palladium(II), dichloro[(S)-(−)-2,2′-bis(diphenylphosphino)-1,1′-binaphthyl]palladium(II), dichloro[(R)-(+)-2,2′-bis(diphenylphosphino)-1,1′-binaphthyl]palladium(II), dichloro 1,1′-bis(diphenylphosphino)ferrocene palladium (II) dichloromethane, dichloro(1,2-bis(diphenylphosphino)ethane)palladium(II), dichloro(1,3-bis(diphenylphosphino)propane)palladium(II), dichloro[1,4-bis(diphenylphosphino)butane]palladium(II), dichloro[9,9-dimethyl-4,5-bis(diphenylphosphino)xanthene]palladium(II), dibromo[1,1′-bis(diphenylphosphino)ferrocene]palladium(II), [(1,2,3-η)-2-Buten-1-yl]chloro[dicyclohexyl(2′,6′-dimethoxy[1,1′-biphenyl]-2-yl)phosphine-κP]palladium, [(1,2,3-q)-2-Butenyl]chloro(tricyclohexylphosphine)palladium, or trans-Dichlorobis(tricyclohexylphosphine)palladium(II).

4. The compound of claim 1, wherein the catalyst is diacetate[(S)-(−)-2,2′-bis(diphenylphosphino)-1,1′-binaphthyl]palladium(II) or dichloro[(S)-(−)-2,2′-bis(diphenylphosphino)-1,1′-binaphthyl]palladium(II).

5. A compound of the formula, 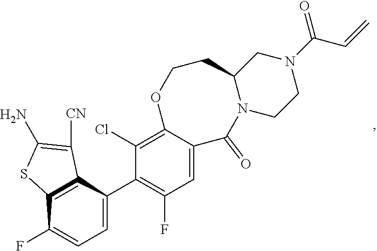
or a pharmaceutically acceptable salt thereof,
obtainable by combining acryloyl chloride and 4-[(13aS)-10-chloro-8-fluoro-6-oxo-2,3,4,12,13,13a-hexahydro-1H-pyrazino[2,1-d][1,5]benzoxazocin-9-yl]-2-amino-7-fluoro-benzothiophene-3-carbonitrile M atropisomer, 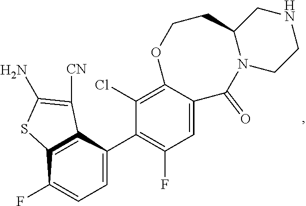
in a continuous stirred-tank reactor, wherein Michael adduct impurity, 4-[(13aS)-2-[3-[(13aS)-9-(2-amino-3-cyano-7-fluoro-benzothiophen-4-yl)-10-chloro-8-fluoro-6-oxo-1,3,4,12,13,13a-hexahydropyrazino[2,1-d][1,5]benzoxazocin-2-yl]-3-oxo-propyl]-10-chloro-8-fluoro-6-oxo-1,3,4,12,13,13a-hexahydropyrazino[2,1-d][1,5]benzoxazocin-9-yl]-2-amino-7-fluoro-benzothiophene-3-carbonitrile, M,M atropisomer, 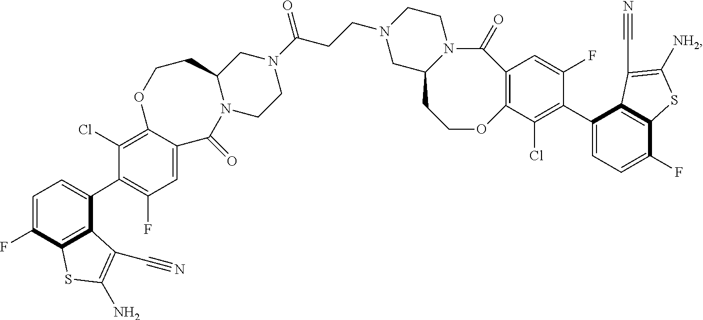
is less than 1% as measured by HPLC Analysis.

6. A compound of the formula, 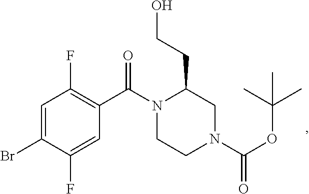 [SMILES: CC(C)(C)OC(=O)N1CCN(C(=O)c2cc(F)c(Br)cc2F)[C@@H](CCO)C1]
or a pharmaceutically acceptable salt thereof.

7. The compound of claim 6, wherein the compound is

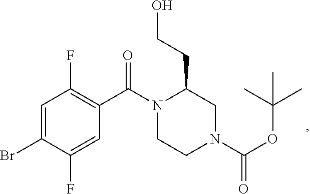 [SMILES: CC(C)(C)OC(=O)N1CCN(C(=O)c2cc(F)c(Br)cc2F)[C@@H](CCO)C1]

8. A compound of the formula, 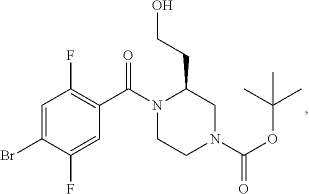 [SMILES: CC(C)(C)OC(=O)N1CCN(C(=O)c2cc(F)c(Br)cc2F)[C@@H](CCO)C1]
or a pharmaceutically acceptable salt thereof,
obtainable by combining tert-butyl (3S)-3-(2-hydroxyethyl)piperazine-1-carboxylate:phosphoric acid (1:1), a base, and 4-bromo-2,5-difluorobenzoic acid, to give tert-butyl (3S)-4-(4-bromo-2,5-difluoro-benzoyl)-3-(2-hydroxyethyl)piperazine-1-carboxylate.

9. A compound of the formula, 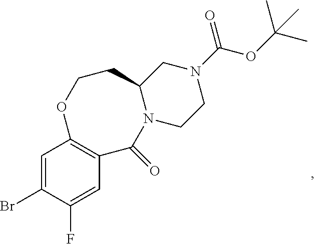 [SMILES: CC(C)(C)OC(=O)N1CCN2C(=O)c3cc(F)c(Br)cc3OCC[C@H]2C1]
or a pharmaceutically acceptable salt thereof.

10. The compound of claim 9, wherein the compound is,

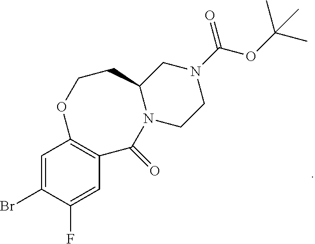 [SMILES: CC(C)(C)OC(=O)N1CCN2C(=O)c3cc(F)c(Br)cc3OCC[C@H]2C1]

11. A compound of the formula, 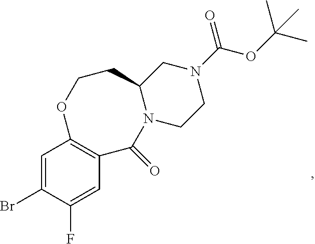 [SMILES: CC(C)(C)OC(=O)N1CCN2C(=O)c3cc(F)c(Br)cc3OCC[C@H]2C1]
or a pharmaceutically acceptable salt thereof,
obtainable by combining tert-butyl (3S)-4-(4-bromo-2,5-difluoro-benzoyl)-3-(2-hydroxyethyl)piperazine-1-carboxylate with a cyclization base to give tert-butyl (13aS)-9-bromo-8-fluoro-6-oxo-1,3,4,12,13,13a-hexahydropyrazino[2,1-d][1,5]benzoxazocine-2-carboxylate.

12. The compound of claim 11, wherein the cyclization base comprises sodium hydride, N,N-diisopropylethylamine (DIPEA), triethylamine (TEA), cesium carbonate, diazabicycloundecene (DBU), sodium tert-butoxide, sodium tert-pentoxide, sodium tert-amylate, potassium tert-pentoxide, or potassium tert-butoxide.

13. The compound of claim 11, wherein the cyclization base comprises potassium tert-butoxide, sodium tert-amylate, potassium tert-pentoxide, sodium tert-butoxide, or sodium tert-pentoxide.

14. The compound of claim 11, obtainable by further comprising a cyclization solvent.

15. The compound of claim 14, wherein the cyclization solvent comprises DMF, NMP, DMAc, DMSO, or THF.

16. A compound of the formula, 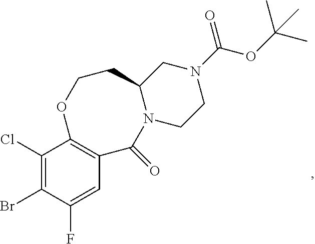 [SMILES: CC(C)(C)OC(=O)N1CCN2C(=O)c3cc(F)c(Br)c(Cl)c3OCC[C@H]2C1]
or a pharmaceutically acceptable salt thereof,
obtainable by combining tert-Butyl (13aS)-9-bromo-8-fluoro-6-oxo-1,3,4,12,13,13a-hexahydropyrazino[2,1-d][1,5]benzoxazocine-2-carboxylate with a chlorinating agent to give tert-Butyl (13aS)-9-bromo-10-chloro-8-fluoro-6-oxo-1,3,4,12,13,13a-hexahydropyrazino[2,1-d][1,5]benzoxazocine-2-carboxylate.

17. A pharmaceutical composition comprising a compound according to claim 1, or a pharmaceutically acceptable salt thereof, and a pharmaceutically acceptable carrier, diluent or excipient.

18. A method of treating a patient for cancer wherein one or more cells express KRas G12C mutant protein, comprising administering to a patient in need thereof, an effective amount of a pharmaceutical composition according to claim 17, wherein the cancer is selected from lung cancer, advanced non-small cell lung cancer, pancreatic cancer, cervical cancer, esophageal cancer, endometrial cancer, ovarian cancer, cholangiocarcinoma, and colorectal cancer.

19. A method of treating a patient for cancer wherein one or more cells express KRas G12C mutant protein, comprising administering to a patient in need thereof, an effective amount of a compound according to claim 1, or a pharmaceutically acceptable salt thereof, wherein the cancer is selected from lung cancer, advanced non-small cell lung cancer, pancreatic cancer, cervical cancer, esophageal cancer, endometrial cancer, ovarian cancer, cholangiocarcinoma, and colorectal cancer.

20. The method according to claim 19 wherein the cancer is non-small cell lung cancer.

21. The method according to claim 19, wherein the cancer is advanced non-small cell lung cancer.

22. The method according to claim 19 wherein the cancer is colorectal cancer.

23. The method according to claim 19 wherein the cancer is pancreatic cancer.

24. The method according to claim 19 wherein the patient has a cancer that was determined to have one or more cells expressing the KRas G12C mutant protein prior to administration of the compound or a pharmaceutically acceptable salt thereof.

25. A method of treating a patient with a cancer that has a KRAS G12C mutation comprising administering to the patient in need thereof an effective amount of a compound according to claim 1, or a pharmaceutically acceptable salt thereof.

26. The method according to claim 19, wherein the patient is also administered an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.

27. The method according to claim 19, wherein the patient has not received prior therapy with a KRAS G12C inhibitor, or a pharmaceutically acceptable salt thereof.

28. The method according to claim 19, wherein the patient has not received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.

29. The method according to claim 19, wherein the patient received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.

30. The method according to claim 19, wherein the patient received prior therapy with a KRAS G12C inhibitor, or a pharmaceutically acceptable salt thereof.

31. The method according to claim 19, wherein the patient has not received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.

32. The method according to claim 19, wherein the patient received prior therapy with an effective amount of one or more of a PD-1 inhibitor, a PD-L1 inhibitor, a CD4/CDK6 inhibitor, or a pharmaceutically acceptable salt thereof, an EGFR inhibitor, or a pharmaceutically acceptable salt thereof, an ERK inhibitor, or a pharmaceutically acceptable salt thereof, a platinum agent, pemetrexed, an Aurora A inhibitor, or a pharmaceutically acceptable salt thereof, a SHP2 inhibitor, or pharmaceutically acceptable salts thereof, or a pharmaceutically acceptable salt thereof.

33. (canceled)

34. (canceled)

35. (canceled)

36. (canceled)

37. A method of preparing a compound of claim 6, the method comprising:
combining tert-butyl (3S)-3-(2-hydroxyethyl)piperazine-1-carboxylate:phosphoric acid (1:1), a base, and 4-bromo-2,5-difluorobenzoic acid, to give tert-butyl (3S)-4-(4-bromo-2,5-difluoro-benzoyl)-3-(2-hydroxyethyl)piperazine-1-carboxylate.

38. A method of preparation of a compound according to claim 9, the method comprising:
combining tert-butyl (3S)-4-(4-bromo-2,5-difluoro-benzoyl)-3-(2-hydroxyethyl)piperazine-1-carboxylate with a cyclization base to give tert-butyl (13aS)-9-bromo-8-fluoro-6-oxo-1,3,4,12,13,13a-hexahydropyrazino[2,1-d][1,5]benzoxazocine-2-carboxylate.

39. The method of claim 38 wherein the cyclization base comprises sodium hydride, N,N-diisopropylethylamine (DIPEA), triethylamine (TEA), cesium carbonate, diazabicycloundecene (DBU), sodium tert-butoxide, sodium tert-pentoxide, sodium tert-amylate, potassium tert-pentoxide, or potassium tert-butoxide.

40. The method of claim 38, wherein the cyclization base comprises potassium tert-butoxide, sodium tert-amylate, potassium tert-pentoxide, sodium tert-butoxide, or sodium tert-pentoxide.

41. The method of claim 38, wherein the method further comprises a cyclization solvent.

42. The method of claim 41 wherein the cyclization solvent comprises DMF, NMP, DMAc, DMSO, or THF.

43. A method of preparing a compound of the formula, 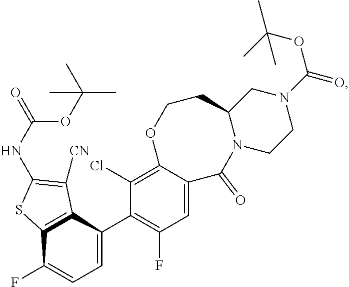
or a pharmaceutically acceptable salt thereof, the method comprising:
reacting tert-butyl (13aS)-9-bromo-10-chloro-8-fluoro-6-oxo-1,3,4,12,13,13a-hexahydropyrazino[2,1-d][1,5]benzoxazocine-2-carboxylate, tert-butyl N-[3-cyano-4-(5,5-dimethyl-1,3,2-dioxaborinan-2-yl)-7-fluoro-benzothiophen-2-yl]carbamate, potassium carbonate, and dichloro[(S)-(−)-2,2′-bis(diphenylphosphino)-1,1′-binaphthyl]palladium(II), in a solvent.

44. A method of preparing a compound of the formula, 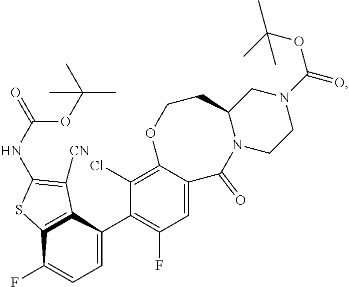
or a pharmaceutically acceptable salt thereof, the method comprising:
reacting tert-butyl (13aS)-9-bromo-10-chloro-8-fluoro-6-oxo-1,3,4,12,13,13a-hexahydropyrazino[2,1-d][1,5]benzoxazocine-2-carboxylate, diacetate[(S)-(−)-2,2′-bis(diphenylphosphino)-1,1′-binaphthyl]palladium(II), tert-butyl N-[3-cyano-4-(5,5-dimethyl-1,3,2-dioxaborinan-2-yl)-7-fluoro-benzothiophen-2-yl]carbamate, a base, and 1,1,1-tris(hydroxymethyl)ethane.
